(19) United States
(12) Patent Application Publication
(10) Pub. No.: US 2011/0142719 A1
(43) Pub. Date: Jun. 16, 2011

(54) CARBON MONOXIDE (CO) MICROSIR SENSOR SYSTEM

(75) Inventors: Mark K. Goldstein, Del Mar, CA (US); Michelle S. Own, Chula Vista, CA (US)

(73) Assignee: Quantum Group Inc.

(21) Appl. No.: 13/037,094

(22) Filed: Feb. 28, 2011

Related U.S. Application Data
(63) Continuation of application No. 11/786,883, filed on Apr. 13, 2007.
(60) Provisional application No. 60/792,103, filed on Apr. 13, 2006.

(51) Int. Cl. G01N 33/00

(52) U.S. Cl. 422/83

(57) ABSTRACT

The present invention provides very small low cost apparatus and method for determining the concentration and/or hazard from a target gas by means of optically monitoring one or more sensors that responds to carbon monoxide. The apparatus comprises a photon source optically coupled to the sensor and the photon intensity passing through the sensor is quantified by one or more photodiode(s) in a system, so that the photon flux is a function of at least one sensor's response to the target gas, e.g., transmits light through the sensor to the photodiode. The photocurrent from the photodiode is converted to a sensor reading value proportional to the optical characteristics of the sensors and is loaded into a microprocessor or other logic circuit. In the microprocessor, the sensor readings may be differentiated to determine the rate of change of the sensor readings and the total photons absorbed value may be used to calculate the CO concentration.

### CROSS-REFERENCE TO RELATED APPLICATION(S)

This application is a continuation of application Ser. No. 11/786,883 filed Apr. 13, 2007, which claimed the benefit of the filing date of U.S. Provisional Application No. 60/792,103 filed Apr. 13, 2006, the disclosures of which are incorporated fully herein by reference.

### FIELD OF THE INVENTION

The present invention relates to improvements for detecting the presence of carbon monoxide by means of one instead of two solid-state sensing elements such as the chemical complexes coated onto porous substrates to produce CO sensors, which was previously described in an earlier invention U.S. Pat. No. 5,618,493, which discloses a means for detecting carbon monoxide sensors, which met UL 2034 but used two sensing elements to do that because one could pass by itself after UL changed the standard in 1995. The single sensor is smaller and less expensive, yet out performs the larger dual sensing system. The single (sensing element) sensor is integrated into a humidity and air quality control device, which regulates the humidity in the micro-environment of the housing the sensing element and the air diffusing from the outside passes through some small holes and then through a getter system that removes basic gases and vapors as well as other compounds that could react with the sensor. The amount of materials used to make the MICROSIR sensor is 40 times less than the SIR sensors, and thus lower in material cost.

### BACKGROUND OF THE INVENTION

Gases and vapors such as carbon monoxide, and other reducing agents can be detected by a single rather two substrate formulation of the invention. These compounds are difficult to detect accurately (plus or minus 5%) without expensive technology such as instruments costing over $100 to $100,000 depending upon the accuracy and type of technology used. Carbon monoxide (CO) has no smell, cannot be seen or tasted, but is very toxic. Such gases are hazardous to humans in automobiles, airplanes, mines, residential and commercial buildings, and other environments in which humans live, work or spend time.

For many years various chemical sensors have been used to detect the presence of toxins. For example, the use of palladium and molybdenum salts for carbon monoxide detection is described in Analytical Chemistry, Vol. 19, No. 2, pages 77-81 (1974). Later, K. Shuler and G. Schrauzer improved upon this technology by adding a third metallic salt component, which produces a self-regenerating catalyst that is short-lived. The catalyst, disclosed in U.S. Pat. No. 4,043,934, uses the impregnation of a carbon monoxide-sensitive chemical catalyst solution into powdered silica-gel substrates to give detectors sensitivity to low concentrations of atmospheric carbon monoxide. While this system is effective in detecting carbon monoxide, it has not met with commercial acceptance due to the short functional life of the catalyst.

It is generally recognized that, for a carbon-monoxide sensor system to be commercially useful, it must have a functional life of at least one year and, preferably 5 to 10 years. Tests have shown that the material described in U.S. Pat. No. 4,043,934 has a working life of only two to four months at room temperature and only three to four days at forty degrees Celsius (40° C.).

U.S. Pat. No. 5,063,164 provided a method for detecting CO, which has a functional life of at least six years without calibration. However, these formulations, which used only one solid-state substrate does not provide adequate sensitivity under high humidity and high temperature conditions, which cannot resist false alarm limits as specified in the Underwriters Laboratories (UL) 2034.

U.S. Pat. No. 5,618,493 is an improvement over U.S. Pat. Nos. 5,063,164 and 4,043,934. U.S. Pat. No. 5,618,493, which discloses a means for detecting carbon monoxide sensors, which met UL 2034 effective April of 1992 and October of 1995. U.S. Pat. No. 5,618,493, which discloses a means for detecting carbon monoxide sensors, which met UL 2034 effective April of 1992 and October of 1995. Hereafter these above patents are incorporated by reference. U.S. Pat. No. 5,618,493 requires two solid-state bio-derived organometallic complexes coated onto a transparent porous silica substrate to produce CO sensors in order to satisfy the performance requirement listed under UL 2034. The yellow solid-state bio-derived organometallic sensor detects CO well at ambient to low humidity conditions while the red one detects CO at ambient to high humidity conditions.

U.S. Pat. No. 5,618,493 by itself failed to meet the stringent sequential test requirements specified by the 2nd. edition of UL 2034, which became effective Oct. 1, 1998. A new invention was made by Mark Goldstein, U.S. Pat. No. 6,251,344 issued on Jun. 26, 2001, hereafter will be incorporated by reference, was made to better control the humidity and remove potential interference chemicals, which might damage the sensor's sensitivity to CO. November of 2003, Goldstein and Oum made additional improvements to U.S. Pat. No. 6,251,344,131, which described a means to further maintain relative humidity and certain air quality contaminates within a predetermined range for a predetermined period of time within a chamber, which is connected to the atmosphere. The objective is to maintain a specific air quality including relative humidity (RH) within a predetermined range for extended period of time under real world conditions as well as extreme conditions. The controlled chamber(s) is contained within a housing that has one or more small openings to the atmosphere. The relative humidity control system also comprises at least one opening to a reservoir of chemicals including a salt with water in at least some solid or a solution containing at least some excess solid phase salt. This control system maintains predetermined RH % range within the “Controlled Chamber” for a given temperature range regardless of the humidity variations in the outside environment, even allowing operation in a condensing. Such a device is referred as “reservoir,” hereafter. The reservoir allows the sensor formulations disclosed in U.S. Pat. No. 5,618,493 to meet the stringent sequential tests as required by the 2nd. Edition of UL 2034 by maintaining the humidity inside the micro-environment surrounding the sensors as close to ambient condition as possible. The controlled humidity condition prolongs the life of the sensors as they are subjected to extreme test conditions ranging from −40° C. to +70° C. and from 15% RH to 95% RH sequentially without having to the replace any sensors from start to finish over a period of several months. Although reservoir adds significant cost to manufacturing of the CO detectors, it is much needed in order to meet the UL 2034 requirements and to protect humans (For extended periods of time such as 5 to 10 years).

### SUMMARY OF THE INVENTION

The present invention eliminates the need for two sensing disks by the new chemical formulations of the chemistry as described in detail below. The chemistry was reformulated using a single micro- or mini-size sensing disk. The invention involves new formulations of sensing chemistry, specially combined and optimized so that only ONE instead of TWO sensing elements is enough to meet the requirement specified under UL 2034. The new single sensing chemistry formulations have been proven to perform better than both the regular-sized SIR sensors in the SIR assembly. The micro-sized porous silica substrates are similar in composition but slightly different in pore diameter and structure. The regular-sized substrates are ˜0.100″ diameter×˜0.050, 0.100, 0.150, and 230″ thick and the micro-sized substrates are ˜0.100 diameter×˜0.025″ and 0.050″ thick. The new sensing chemistry formulations can be applied to the substrates by either the injection or the immersion method. The injection method eliminates waste.

There are a number of methods to compute the CO hazard and these is subject of another patent to be filed. In addition, a preferred method to meet the BSI and European CO Standards is described using two sensor systems with two different sensors each having different sensitivity within one housing. The single housing dual sensor uses one LED and two photodiodes. The novel two sensors method to meet the European (BSI) CO standard is similar to the method developed to meet the Japanese standard.

The major advantages of MICROSIR over SIR are: 1. Lower cost (estimates saving of US $1.25 per sensor, 2. Better controlled gas path therefore more accurate and more precision, 3. Better getter system therefore longer life (as shown by ammonia accelerated age tests), and 4. Better RESERVOIR SYSTEM THEREFORE BETTER humidity CONTROL AT BOTH LOW AND HIGH (as shown by sensor response curves). 5. The MICROSIR Edgeview is faster and meets the Japanese standard for CO and the European Standard for CO enhanced smoke, 6. More easily automated as the board of alarms use surface mount and MICROSIR is a surface mount part that attaches over surface mounted optics after the soldering, 7. small size, and 8. approved UL recognized component.

The MICROSIR device can also be used to detect the CO, which may be combined with temperature and smoke in a very small package. The detection of one or more indicators such as smoke and CO; increases the sensitivity of the other indicators. Combining signals produces an improved fire detector comprising a CO sensor and a smoke sensor in one unit. The smoke detection sensor may be either ionization or photoelectric either or both may be combined with the CO sensor to provide earlier warning to fire and reduce false alarms.

The new single CO sensing element can replace the “dual CO sensing element in the current SIR CO alarm when the regular-size substrates are used. However, it requires UL approval testing from all over again.

The time and money it takes to get UL approval for switching from a dual to a single regular-sized CO sensing element is better justified when the single CO sensing chemistry is based on micro- or mini-sized substrates in MICROSIR; however, either size works well and passes all tests.

The new invention reduces the cost of sensor manufacturing by eliminating the need for two sensing disks as well as by miniaturizing the sensing disk to require only 1/10 to 1/20 of the current starting materials. The miniaturized single-sensing element requires less than 1/10 of the reservoirs materials (plastic, membrane, and chemical content). Bottom line, the new invention is expected to yield a net saving of 30 to 50% of the current manufacturing cost while exceeding or at least maintaining the same or better performance as the current SIR CO sensors which required TWO regular sized sensing elements. The Single-Sensing Micro-SIR has been shown to meet the latest UL 2034 for residential, recreational vehicles and boats applications.

Like the dual sensing elements counterpart, the new sensing element also needs reservoirs in order to meet the current UL 2034.

Here are a few examples of applications for the new Single-Sensing-SIR:

1. CO Alarms for Residential, Commercial, and Recreational Applications

As mentioned above, the new Single-Sensing-SIR has been shown to meet the UL 2034 and 2075 for protecting human life against CO poisoning at homes, in commercial buildings, as well as in recreational vehicles and boats.

2. Visual CO Indicator

The new invention can be used as a visual CO detector for detecting the presence of CO. As visual CO detectors, the sensors made according to the formulations according to this invention, requires no power, no electronic, nor software. In the presence of CO, the sensor changes from tan-orange to dark-blue at about 5-10% COHb. In the absence of CO, the sensors self-regenerate within a few hours to its original color and are reusable. These sensors also have over 6 years of operational life compared to 3 months for other technologies such as AIR-ZONE and DEAD-STOP. In addition to their amazing long sensor life, they also outperformed both AIR-ZONE and DEAD-STOP under wider range of relative humidity and temperature.

3. Digital CO Alarms and/or CO Instrumentation

Results have indicated that the new Single-Sensing-SIR offers real potential for designing and manufacturing reliable, low cost CO alarms and potentially CO analyzers that allows digital display of the CO concentration on liquid crystal display (LCD).

4. CO Sensing for Fuel Cell Applications

In addition, the present invention can also be further modified with extra copper ions to better detect carbon monoxide in the presence of high concentration of hydrogen and other gases commonly found in fuel cells. These formulations are called the K sensor series and are a subject of co-pending application, “Carbon Monoxide Control System,” U.S. patent application Ser. No. 09/965,105; Filed Sep. 26, 2001; and hereafter will be incorporated by reference. There exists a real need for a CO sensing system capable of high CO selectivity and stability for use in fuel cells applications.

5. CO to CO₂ Conversion for Fuel Cell Applications

The K formulations are also excellent catalyst for converting CO to CO₂ even in the absence of oxygen for a period of time. This is a subject of co-pending application, co-pending application, “Carbon Monoxide Control System,”; U.S. patent application Ser. No. 09/965,105; Filed Sep. 26, 2001 and “Improved CO Catalyst System to Remove CO,” U.S. patent application Ser. No. 11/058,132 Filed Feb. 14, 2005 and herein will be incorporated by reference.

A carbon monoxide sensor system prepared according to principles of this present invention is single chemical sensing element for CO enclosed within a sensor housing, which is further contained with a chemical reservoir, which is subject of a co-pending patent application titled, “Chemical System for Controlling Relative Humidity and Air Quality,” U.S. patent application Ser. No. 10/997,646, filed Nov. 24, 2004. Component of the single chemical sensing element is manufactured from a porous solid-state material (substrate), which is sufficiently transmissive to light to permit detection of the transmitted light through the sensor by human eye or by a photodiode or the like. A substrate is coated with various types of chemical reagent mixtures that are formulated from combining and optimizing the low humidity CO sensing reagent (yellow) with the high humidity (red) CO sensing reagent disclosed in U.S. Pat. No. 5,618,493 in desired ratios to reduce a certain percentage of light transmittance through the sensor in relation to an increase in carbon monoxide concentration in the air. These hybrid sensors are called the S6 and S66 sensor series. These sensors are well suited for detecting CO in the range of 30 to 1,000 ppm. KY sensor series are better suited for detecting greater than 1,000 ppm CO. The S6 and S66 and KY sensor series re-gain their light transmittance the present of clean air (CO concentration <5 ppm). Any combinations of the three sensor series of S6, S66, and KY to form a TWO elements sensing system are referred to as an S34 series.

Additional new CO sensing formulations that have increased sensitivity to CO after having been stored in low relative humidity for an extended period of time are referred to as the MO37-32 series.

The MICROSIR is small with a very low profile make it suitable for many application where small size is desired. The MICROSIR has at least 6 additional advantages over the current SIR sensor system. These advantages of MICROSIR over SIR are:

1. Lower cost (estimates saving of US $1.25 per sensor,

2. Better controlled-gas-path, therefore more accurate and more precision,

3. Better getter system therefore longer life (as shown by ammonia accelerated age tests), and

4. Better RESERVOIR SYSTEM THEREFORE BETTER Humidity CONTROL AT BOTH LOW AND HIGH (as shown by sensor response curves).

5. The MICROSIR Edgeview is faster and meets the Japanese standard for CO and the European Standard for CO enhanced smoke, and

6. More easily automated as the board of alarms use surface mount and MICROSIR is a surface mount part that attaches over surface mount optic after soldering.

### BRIEF DESCRIPTION OF THE DRAWINGS

FIG. 1 is an assembly drawing of MICROSIR MOD3-01 system 100 with only ONE, mini-sized CO sensing element located inside a MICROSIR reservoir assembly.

FIG. 2 is an assembly drawing of MICROSIR MOD3-02 system 200 with TWO, mini-sized CO sensing elements located inside a MICROSIR reservoir assembly.

FIG. 3 is an assembly drawing of SIR-01 system 300 with ONE, standard-sized CO sensing element located inside a regular SIR reservoir assembly.

FIG. 4 is an assembly drawing of SIR-02 system 400 with TWO, standard-sized, CO sensing elements in regular SIR reservoir assembly.

FIG. 5 is a plot of digital display of ppm CO versus time in 70 ppm CO test.

FIG. 6 are a plot of digital display of ppm CO versus time in 150 ppm CO test.

FIG. 7 is an assembly drawing of MICROSIR MOD1-01 system with only ONE, mini-sized CO sensing element located inside a MICROSIR reservoir assembly.

FIG. 8 is an assembly drawing of MICROSIR MOD1-02 system with TWO, mini-sized CO sensing elements located inside a MICROSIR housing assembly.

FIG. 9 is a side-view illustration of the theory of operation for the MICROSIR CO sensing system.

FIG. 10 is graphical representation showing response characteristics of a ONE mini-sized CO sensor type S66 in a MICROSIR MOD1-01 to 70 ppm 1002, 150 ppm 1003, and 400 ppm CO 1004 at 23±3° C. and 55±5% RH, as specified in criteria 1.

FIG. 11A is graphical representation showing response characteristics of the same MICROSIR CO sensor system from FIGS. 10 to 30 ppm 11A01, 70 ppm 11A02, 150 ppm 11A03, and 400 ppm CO 11A04 at 49° C. and 40% RH, as specified in criteria 6.

FIG. 11B is graphical representation showing response characteristics of the same MICROSIR CO sensor system from FIG. 11A to 70 ppm 11B02, 150 ppm 11B03, and 400 ppm CO 11B04 at 66° C. and 40% RH, as specified in UL 2034 Section 69.1a.

FIG. 12A is graphical representation showing response characteristics of the same MICROSIR CO sensor system from FIG. 11B to 30 ppm 12A01, 70 ppm 12A02, 150 ppm 12A03, and 400 ppm CO 12A04 at 0° C. and 15% RH, as specified in Criterion 7 or UL 2034 Section 45.1.

FIG. 12B is graphical representation showing response characteristics of the same MICROSIR CO sensor system from FIG. 12A to 30 ppm 12B01, 70 ppm 12B02, 150 ppm 12B03, and 400 ppm CO 12B04 at minus (−) 40° C., as specified in UL 2034 Section 69.1b.

FIG. 13 is graphical representation showing response characteristics of the same MICROSIR CO sensor system from FIG. 12B to 30 ppm 1301, 70 ppm 1302, 150 ppm 1303, and 400 ppm CO 1304 at minus 61° C. and 93% RH, as specified in UL 2034 Section 69.1c.

FIG. 14 is graphical representation showing response characteristics of the same MICROSIR CO sensor system from FIGS. 13 to 30 ppm 1401, 70 ppm 1402, 150 ppm 1403, and 400 ppm CO 1404 at minus 23° C. and 10% RH, as specified in UL 2034 Section 46A.2.

FIG. 15A is graphical representation showing comparative response characteristics of ONE mini-sized CO sensor from the S66 sensor series to 150 ppm CO in a MICROSIR MOD1-01 15A1 versus in a MICROSIR MOD3-01 15A3 at 23±3° C. and 55±5% RH.

FIG. 15B is graphical representation showing comparative response characteristics of TWO mini-sized CO sensing elements from the S34 sensor series to 150 ppm CO in a MICROSIR MOD1-02 15B1 versus in a MICROSIR MOD3-02 15A3 at 23±3° C. and 55±5% RH.

FIG. 16 is graphical representation showing comparative response characteristics of ONE mini-sized CO sensor from the S66 sensor series to 150 ppm CO in a MICROSIR MOD1-01 1601 versus in a MICROSIR MOD3-01 1603 at 66° C. and 40% RH.

FIG. 17 is graphical representation showing comparative response characteristics of ONE mini-sized CO sensor from the S66 sensor series to 150 ppm CO in a MICROSIR MOD1-01 1701 versus in a MICROSIR MOD3-01 1703 at minus (−) 40° C.

FIG. 18 is graphical representation showing IMPROVED response characteristics of the ONE mini-sized S6 formulation with CaCl₂+ZnCl₂/ZnBr₂ additives to 150 ppm CO in a MICROSIR MOD1-01 1801 at 66° C. and 40% RH following 30 days of preconditioning at same conditions of 66° C. and 40% RH.

FIG. 19 is an illustration showing ONE MICROSIR CO sensing element (1975) positioned in edge-view orientation for increase sensitivity to low CO concentration for aiding in early fire and/or smoke (1903) detection application

FIG. 20 is an illustration for explaining the “Theory of Operation of MICROSIR involving TWO sensing elements positioned in edge-view orientation” for increase sensitivity within a wider range of humidity and temperature.

FIG. 21 is an illustration showing two CO sensing elements (2103 A and B) in center-view orientation between one LED 2101 and two photodiodes 2104 and 2102.

FIG. 22A is an illustration for explaining the “Theory of Operation of SIR-01,” one sensing element 22A30 positioned in center-view orientation” between an LED 22A20 and a Photodiode 22A40.

FIG. 22B is an illustration for explaining the “Theory of Operation of SIR-01,” one sensing element 22B35 is positioned in edge-view orientation” between the LED 22B25 and the Photodiode 22B45.

FIG. 23 is graphical representation showing IMPROVED response characteristics of M1-01e with one S50 single sensing element positioned in an edge-view orientation, in response to CO ramp of 5 ppm CO every 30 seconds, from 0 to 40 ppm CO. It is a proof-of-concept result demonstrating the viability of FIG. 19.

FIG. 24 is graphical representation showing response characteristics of M1-01 and M3-01 with varying amount of acid-coated activated charcoal for removing ammonia from air and/or air containing CO before reaching the sensor. Without the ammonia remover, both SIR and MICROSIR CO sensor are expected to have shorter lifetime.

### DETAILED DESCRIPTION OF THE INVENTION

The present invention relates to an improved chemical sensor, which uses only a single porous, translucent substrate coated with chemical reagents disclosed in U.S. Pat. No. 5,618,493, which is herein incorporated by reference, which is reformulated by mixing the red and yellow sensing reagents, and/or by adding bromide and chloride salts of certain transitional metals and/or by substituting CaCl₂ and/or CaBr₂ with halide salts of Al, Cd, Co, Ce, Cr, Fe, Mn, Ni, Sr, Zn, Sb, Ba, Mg, K, as well as Mg(NO₃)₂, NaBr, NaCl, NaHSO₄, Mg(NO₃)₂, KCO₃, KCl, MgSO₄, and any mixture combinations thereof for detecting gases such as carbon monoxide, hydrogen sulfide, formaldehyde, acetone, mercury vapor, and other similar gases or vapors. The chemical sensor constructed according to the principle of this invention is an improvement over the dual sensor system disclosed in U.S. Pat. Nos. 5,618,493 and 5,063,164.

Unlike, the chemical sensor disclosed in U.S. Pat. No. 5,618,493, light emitted by an IR light emitting diode (LED) passes through only a “single sensing element” (not dual), and is detected by a photo detector (photodiode). When this new chemical sensor is exposed to CO it darkens, thereby reducing the amount of light transmitted. The rate of change of the light transmittance reduction as registered by the photodiode is function of CO concentrations in the air. The light transmittance increases as the sensor regenerates when the CO is removed or reduced from an environment. In short, like the dual sensing system, the single sensing systems also changes their optical properties in such a way as to allow easy detection of their response by visible or infrared radiation, e.g., by means of a light emitting diode (LED) such as a 940 nm LED and a photo detector of the same photodiode and are described in more detailed by Eric Gonzales, et al in U.S. Patent Application No. 60/711,748, filed on Aug. 25, 2005.

The improved single chemical sensor system, which detects carbon monoxide and self-regenerates in air, is fabricated from a semi-transparent silica porous substrate, which is manufactured in house according to U.S. Pat. No. 4,059,658 and several modification thereof and doped with mixed oxides. This sensor is initially tan-orange and turns to dark blue when exposed to CO and performs within best between 11 to 95% relative humidity from −40° C. to +70° C. The reservoir keeps the sensor in a narrow range under most all UL testing conditions as well as all real world conditions.

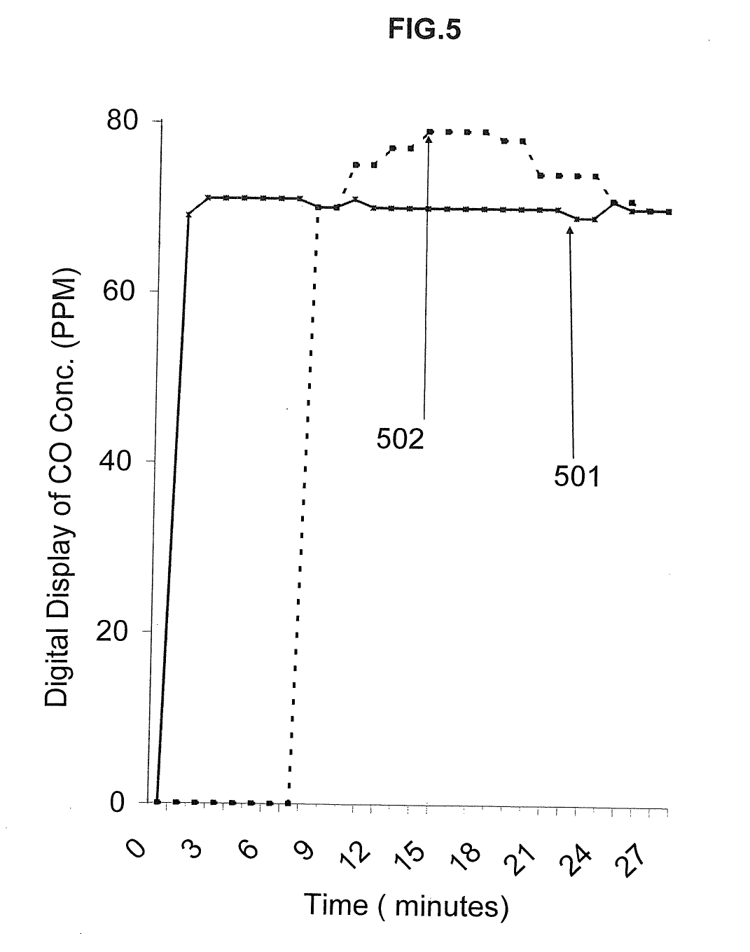

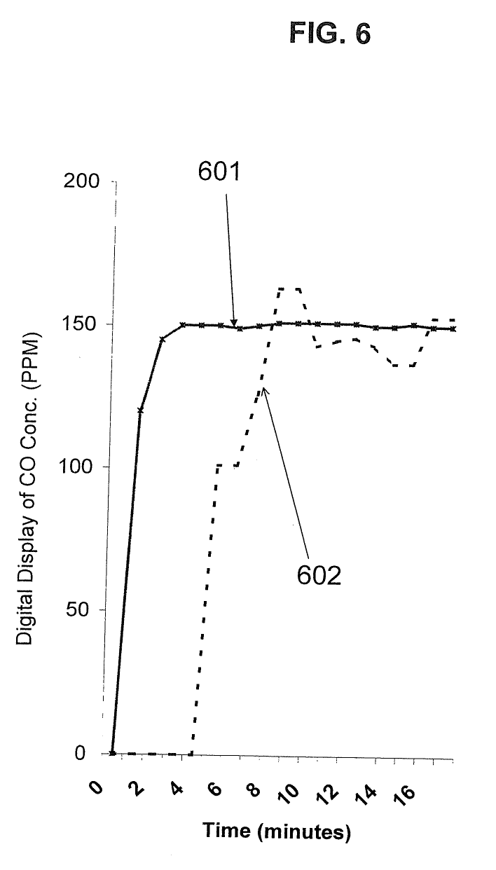

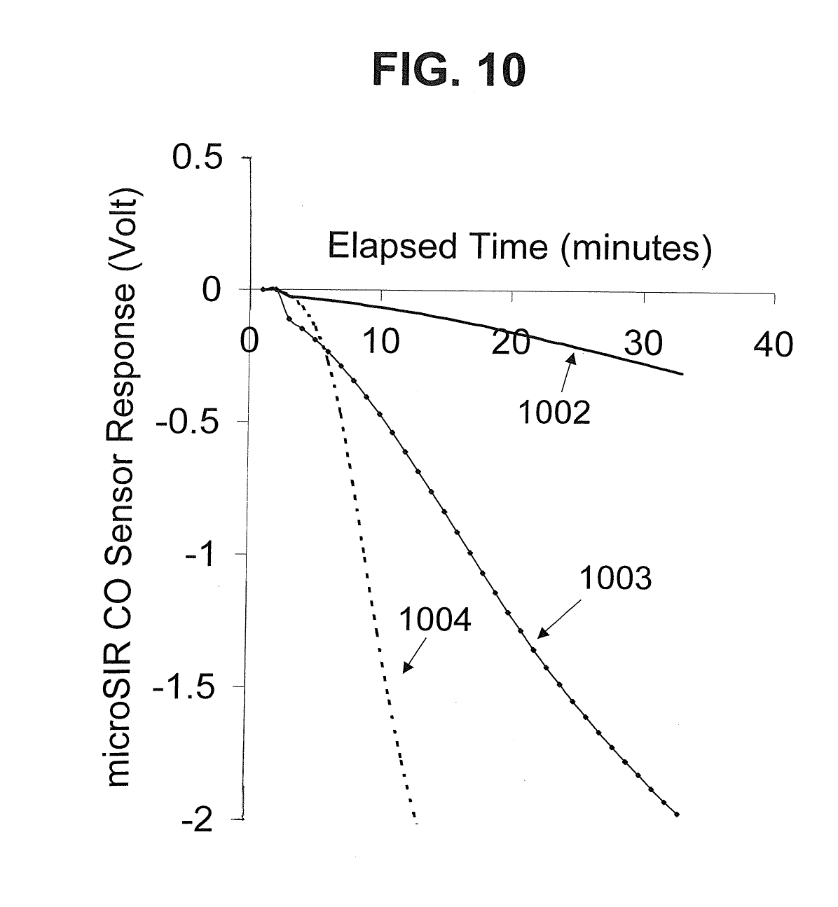

When tested in combination with the new chemical system as described in the, “Improved Chemical System for Controlling Relative Humidity and Air Quality,” U.S. patent application Ser. No. 10/997,646, filed Nov. 24, 2004. This new Single-Sensing Micro-SIR performs well to meet stringent requirement as specified in UL 2034. The results that verify this statement are shown in FIGS. 10 through 14. The Single-Sensing Micro-SIR when combined with the appropriate electronic circuitry and software equations such as those described by Eric Gonzales, et al in the U.S. Patent Application No. 60/711,748, filed on Aug. 25, 2005, also offers real potential for digital CO alarm applications. Preliminary results that demonstrate this capability are shown FIGS. 5 and 6.

The new chemical sensor is made by impregnating a semi-transparent porous silica disk with a chemical mixture, which comprises at least one of the chemical reagents selected from each of the following groups 1 through 8, and further coated onto the porous silica substrates as detailed in groups 9 and 10 and 11:

Group 1: Palladium salts selected from the group consisting of palladium salts of sulfate, palladium sulfite, palladium pyrosulfite, palladium chloride, palladium bromide, palladium iodide, palladium perchlorate, CaPdCl₄, CaPdBr₄, Na₂PdCl₄, Na₂PdBr₄, K₂PdCl₄, K₂PdBr₄, Na₂PdBr₄, CaPdClₓBr_{y}, K₂PdBrₓCl_{y}, Na₂PdBrₓCl_{y} (where x can be 1 to 3 if y is 4 or visa versa), and organometallic palladium compounds such as palladium acetamide tetrafluoroborate and other similarly weakly bound ligands, and mixtures of any portion or all of the above;

Group 2: Molybdenum, vanadium, and/or tungsten salts or acid salts selected from the group consisting of sodium vanadate, silicomolybdic acid, phosphomolybdic acids, and their soluble salts, molybdenum trioxide, ammonium molybdate, alkali metal, or alkaline earth metal salts of the molybdate anions, mixed heteropolymolybdates, and mixtures of any portion or all of the above;

Group 3: Soluble salts of copper halides, sulfates, nitrates, perchlorates, and mixtures thereof, copper organometallic compounds that regenerate the palladium such as copper tetrafluoroacetic acid, copper trifluoroacetylacetonate, and other similar copper compound, and copper vanadium compounds such as copper vanadate, and soluble vanadium compounds that can be incorporated into the group 2 molybdenum based keg ions such as phosphomolybdic acid and silicomolybdic acid, and mixtures of any portion or all of the above;

Group 4: Supramolecular complexing molecules selected from the cyclodextrin family including alpha, beta, and gamma as well as their soluble derivatives such as hydroxymethyl, hydroxyethyl, and hydroxypropyl beta cyclodextrins, crown ethers and their derivative, and mixtures of any portion or all of the above;

Group 5: Soluble salts of alkaline and alkali halides, and certain transitional metal halides such as manganese, cadmium, cobalt, chromium, nickel, zinc, and other soluble halide salts such as AlCl₃, AlBr₃, CdCl₂, CdBr₂, CoCl₂, CoBr₂, CeCl₃, CeBr₃, CrCl₃, CrBr₂, FeCl₃, FeBr₃, MnCl₂, MnBr₂, NiCl₂, NiBr₂, SrCl₂, SrBr₂, ZnCl₂, ZnBr₂, SnCl₂, SnBr₂, BaCl₂, BaCl₂, MgCl₂, MgBr₂, Mg(NO₃)₂, NaBr, NaCl, NaHSO₄, Mg(NO₃)₂, KCO₃, KCl, KBr and/or MgSO₄ and any mixture thereof;

Group 6: Organic solvent and/or co-solvent and trifluorinated organic anion selected from the group including dimethyl sulfoxide (DMSO), tetrahydrofuran (THF), dimethyl formamide (DMF), trichloroacetic acid, sodium salt of trichloroacetic acid, trifluoroacetate, a soluble metal trifluoroacetylacetonate selected from cation consisting of copper, calcium, magnesium, sodium, potassium, lithium, or mixture thereof;

Group 7: Soluble inorganic acids such as hydrochloric acid, sulfuric acid, sulfurous acid, or a mixture thereof;

Group 8: Strong oxidizer such as nitric acid and peroxide, or a mixture thereof.

The mole ratio ranges for the components of the reagent solution mixture used to formulate this new S6 and S66 “single CO sensing element” series for CO detection from 30 to 550 ppm are as follows:

Group 1 Group 3=10.19:1 to 16.98:1

Group 2 Group 3=3.04:1 to 5.07:1

Group 4 Group 3=1.04:1 to 1.74:1

Group 5 Group 3=34.11:1 to 56.84:1

Group 6 Group 3=1.07:1 to 1.79:1

Group 7 Group 3=0.004:1 to 0.04:1

Group 8 Group 3=0.04:1 to 0.08:1

And the mole ratio ranges for the components of the reagent solution mixture used to formulate this new KY “single CO sensing element” for detecting CO ranges from 550 to 10,000-ppm CO are as follows:

Group 2 Group 1=0.20:1 to 0.33:1

Group 3 Group 1=0.10:1 to 4.73:1

Group 4 Group 1=0.05:1 to 0.08:1

Group 5 Group 1=1.75:1 to 2.92:1

Group 6 Group 1=0.00:1 to 0.00:1

Group 7 Group 1=0.62:1 to 1.03:1

Group 8 Group 1=0.70:1 to 1.16:1

The reagent solution mixtures, which contains at least one of the substances selected from groups 1 through 8 above, is further coated onto or encapsulated within a solid porous substrates of at least partial optical transparency to become “Single Sensing Element” for detecting CO. Some of these substrates are listed in Groups 9, 10, and 11 below.

Group 9: Porous silica substrates include, but are not limited to, porous silica gel, porous glass bead, porous silicon dioxide, leached-porous borosilicate, porous metal oxides that are not soluble or do not react with any of the materials in group 1 through 8, and other porous substrates such as those manufactured according to the U.S. Pat. No. 4,059,658 and several modifications thereof. These substrates can be made in many sizes and shapes. Disk-shape is most preferred due to high yield

Group 10: Porous silica substrates from group 9 coated with metal or mixed metal oxides that are not soluble or do not react with any of the chemical reagents described in-groups 1 through 8 such as doped silicon dioxide, CuO, Pr₂O₃, Cr₂O₃, Al₂O₃, Sm₂O₃, ZnO, Yb₂O₃, Er₂O₃, NiO, IrO, CoO, Tm₂O₃, Y₂O₃, ScO, yttria and yttria aluminum garnet (YAG) and mixtures thereof.

Group 11: Porous silica gel such as in bead form, which is commercially available from many suppliers of silica gel or porous silicon dioxide. Such porous silica beads contain average pore diameters ranging from 80 to 150 Angstroms (15 nm) with surface area of 250 to 600 m/gram. An example of this material includes the Grade TS-1 supplied by CHEM SOURCE-EAST, Inc. 7865 Quarterfield Road Severn, Md. 21144, Telephone No. 410-969-3390, which contains bead sizes ranging from 1 to 5 mm., pore diameters range from 110 to 130 angstroms pore, and surface areas range from 340 to 400 m₂/gram surface area, and pore volumes range 0.9 to 1.1 cc/g. These substrates also have performed exceptionally well as substrate support CO oxidation catalysts.

There are many applications for carbon monoxide sensors of this type and therefore there are many preferred embodiments for each of the applications, several of these formulations are described below.

The formulations described below are examples of Single CO Sensing Chemistry types S6 and S66 series on regular-size and mini-sized silica porous substrate (SPS) disks.

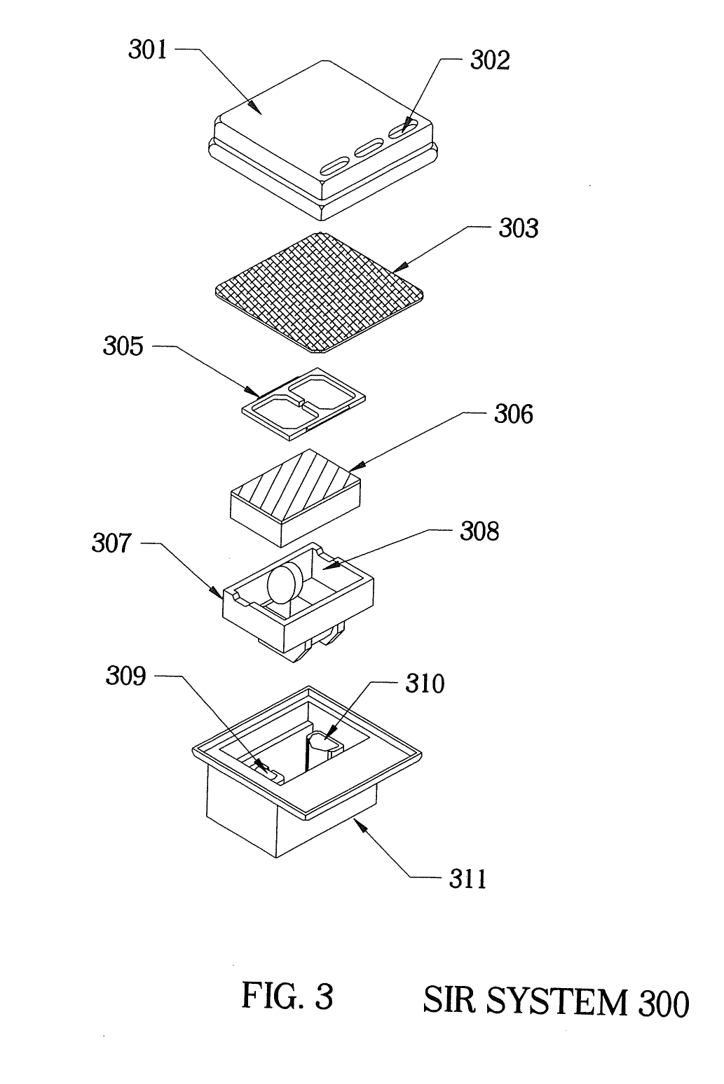

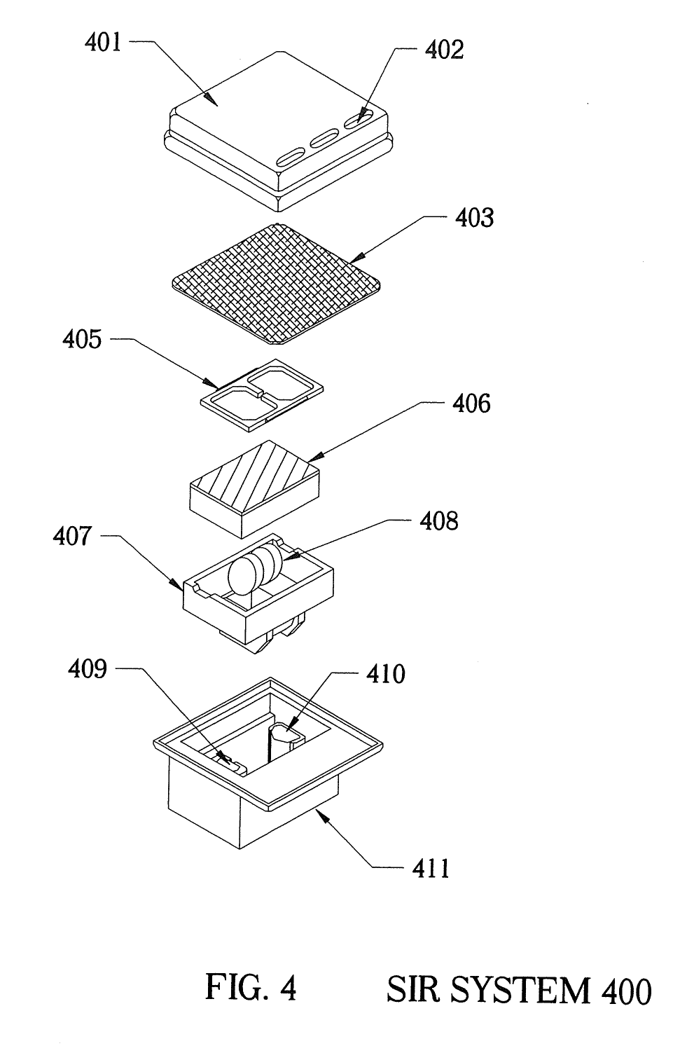

When the regular-sized disks are impregnated with the new hybrid, single CO sensing chemistry, the resulted regular-sized Single CO Sensing elements are to be installed SINGLY inside SIR-01 assembly configuration as shown in FIG. 3. Using, the new reservoir content as detailed in a co-pending patent application, “Improved Chemical System for Controlling Relative Humidity and Air Quality,” U.S. patent application Ser. No. 10/997,646, filed Nov. 24, 2004. The Single Sensing Elements can effectively replace DUAL CO sensing system, hence; reducing cost in the current COSTAR™ CO alarms such as Models 9SIR, 9RV, and 12SIR. However, they must first be improved by UL. A SECOND, regular-sized, CO sensing element type KY series is needed to meet the 550 to 6,000 ppm CO response and recovery requirement for “recreational boats” application under UL 2034. The TWO sensing elements system is referred to as the S34 CO sensor series and to be installed in a SIR-02 assembly configuration as shown in FIG. 4. The S34 comprised any pair of S6 or S66 and KY that provides CO detection range from 30 to 6,000 ppm.

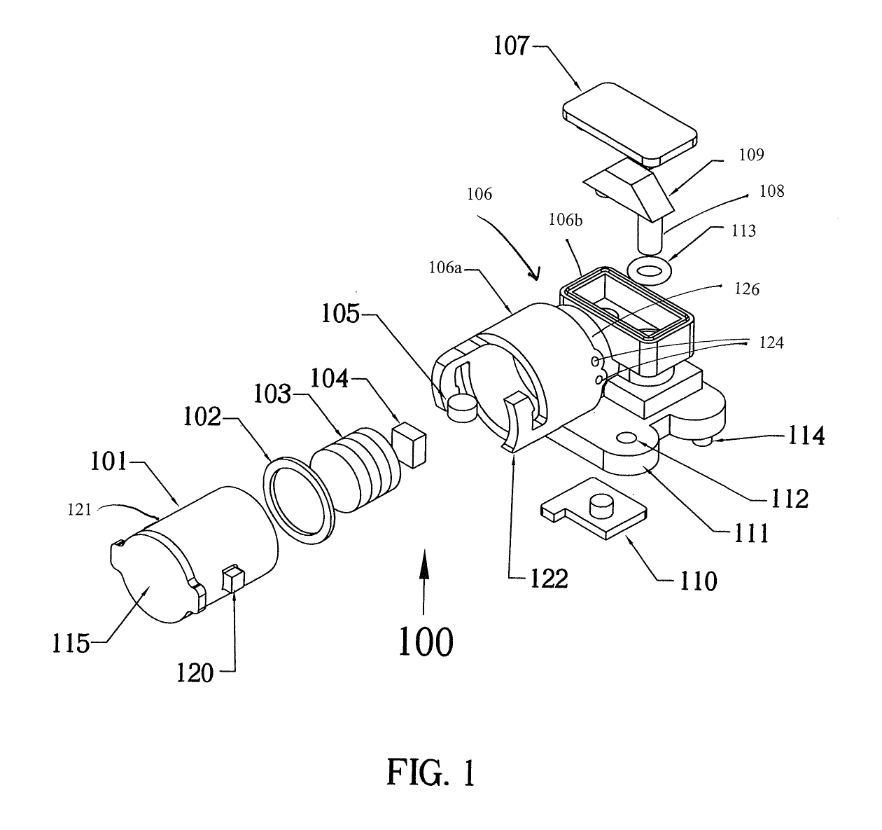

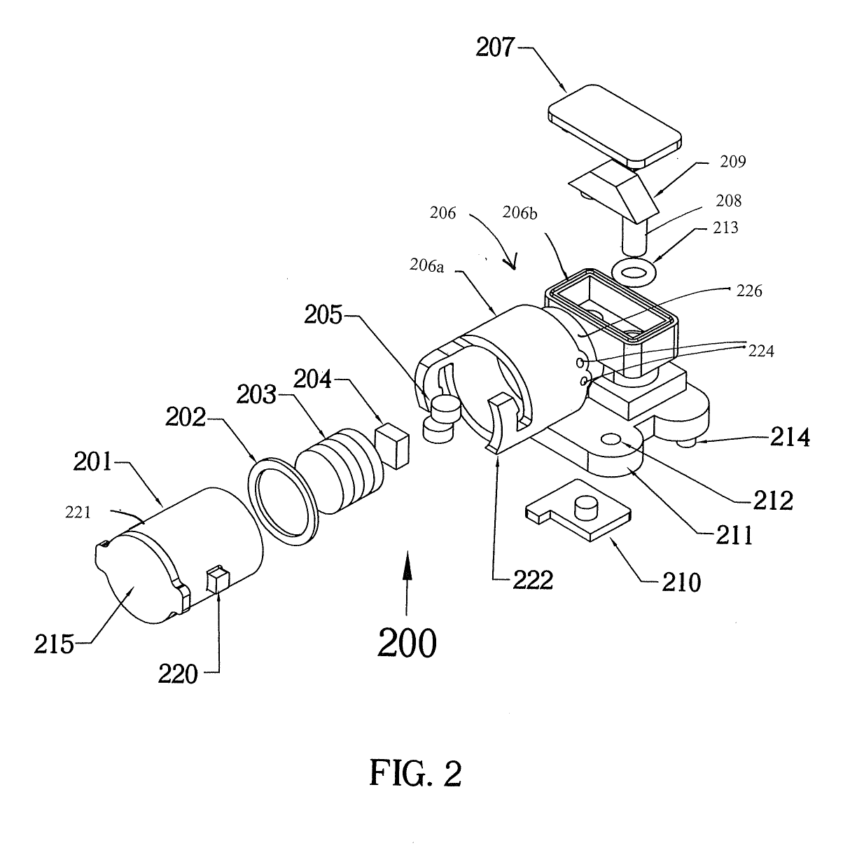

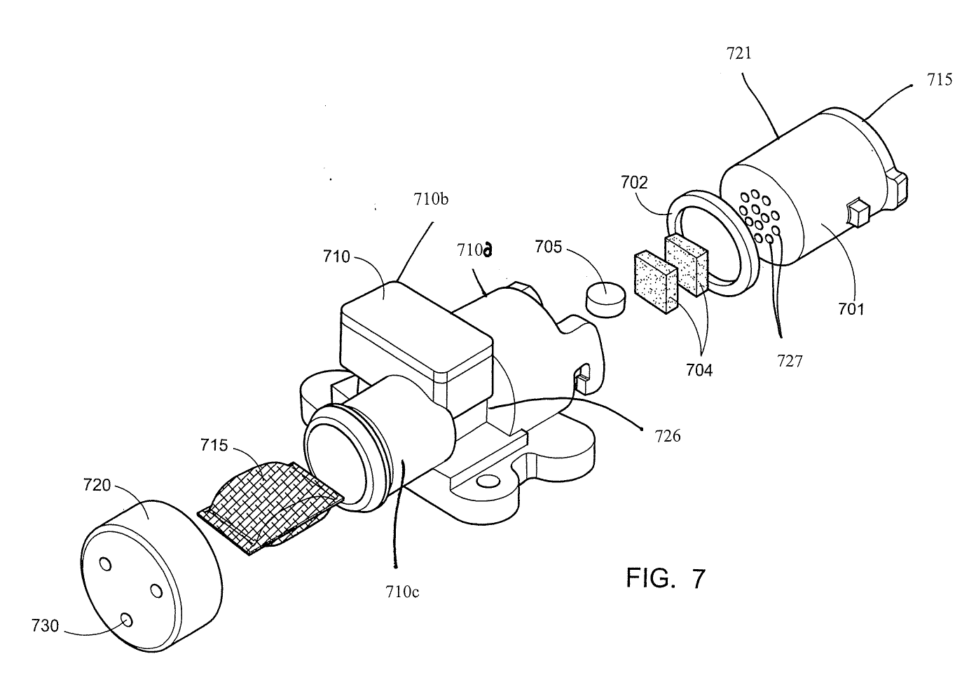

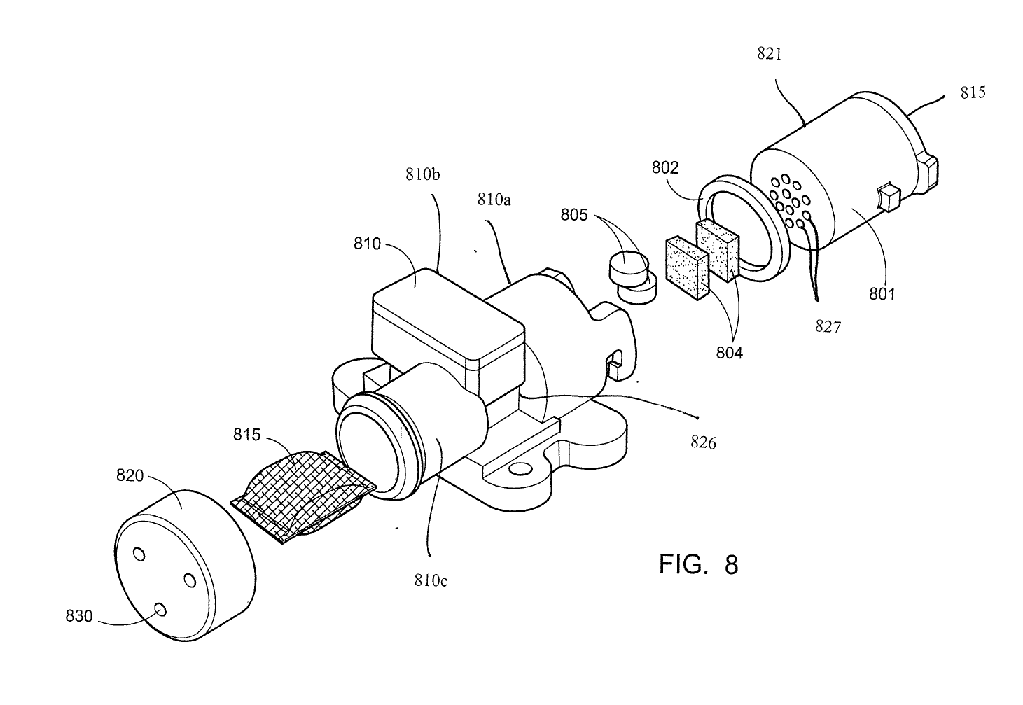

When the mini-sized disks are impregnated with the new single CO sensing chemistry, the resulted mini-SPS Single CO Sensing elements are to be installed SINGLY in the MICROSIR assemblies such as the MOD1-01 (FIG. 7) or the MOD3 (FIG. 1) and tested according to UL 2034 for residential and recreational applications. A SECOND, mini-sized, CO sensing element type KY series is needed to meet the 550 to 6,000 ppm CO response and recovery requirement for “recreational boats” application under UL 2034. The TWO mini-sized sensing elements are referred to as the mini-S34 CO sensor series and to be installed in MICROSIR MOD1-02 (FIG. 8) and MICROSIR MOD3-02 (FIG. 2). The mini-S34 CO sensor series comprised any pair of mins-S6 or mini-S66 and mini-KY; and provides CO detection range from 30 to 6,000 ppm.

“Soak method” is currently used to fabricate the sensors and is described in the examples below. This method unnecessarily wastes 67% of the sensing reagents per standard-size SPS and 72% per mini-sized SPS, when compares to “Injection method.” However, the cost of labor for the “manual injection method” outweighs the cost of the wasted sensing reagents. Future manufacturing of these sensors should be based on an “automated injection method” to save on both labor and material costs.

Either method, SOAK or INJECTION, works for any sensor formulations on standard-size SPS and mini-size SPS. Example 1A and 1B described both methods in details.

### Preferred Embodiment 1

### Visual CO Indicator

### Example 1A

### Single CO Sensing Formulation S6e on Regular-Sized SPS for SIR

“Soak Method”

100 of 0.150″ diameter×0.100″ thick silica porous silicate disks with pore diameter ranging from 200 to 300 angstroms and surface area ranging from 100 to 200 square meter per gram are soaked in a 15-mL of the new S6e sensing formulation containing 7.7 mmole of H₄SiMo₁₂O₄₀.xH₂O, 77.7 mmole of CaCl₂.2H₂O, 2.7 mmole of CCl3COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside an humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

Manual Injection Method

100 of 0.150″ diameter×0.100″ thick silica porous silicate disks with pore diameter ranging from 200 to 300 angstroms and surface area ranging from 100 to 200 square meter per gram are spread flat on a clean Pyrex tray or polyethylene tray.

Using a micropipette, inject 50-microliters of the new S6e sensing formulation containing 7.7 mmole of H₄SiMol₂O40.xH2O, 77.7 mmole of CaCl₂.2H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin on the bottom-side of each disk. The tray is inserted inside a polyester felt pillow case, while sitting inside a humidity and temperature controlled room or chamber with relative humidity maintain within 45 to 55% and temperature within 20 to 26° C. for optimal self-assembly of the supramolecular layering. After 14 to 24 hours, the pillowcase is removed and the sensors are allowed to further air dry in the same controlled room for an additional 14 to 24 hours. Then the sensor tray is placed inside 40° C. drying oven for 14 to 24 hours. Then the sensor tray is removed and stored inside the humidity and temperature controlled chamber. The sensors are now ready for use or for test.

### Preferred Embodiment 2

### Single Sensing Element MICROSIR for CO Alarm that Meets UL 2034

### Example 1B

### Single Sensing Formulation S6e on Mini-SPS for MICROSIR

“Soak Method”

600 of the mini-sized silica porous silicate disks with pore diameter ranging from 200 to 300 angstroms and surface area ranging from 100 to 200 square meter per gram are soaked in a 15-mL of the new S6e sensing formulation containing 7.7 mmole of H₄SiMo₁₂O₄₀.xH₂O, 77.7 mmole of CaCl₂.2H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside an humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

“Injection Method”

Mini-sized SPS are spread flat on a clean Pyrex or plastic tray. 7 to 10 microliters of the single sensing element reagent mixture S6e containing 7.7 mmole of H₄SiMo₁₂O₄₀.xH₂O, 77.7 mmole of CaCl₂.2H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin is injected directly onto a mini-sized porous silica substrate (SPS) having the dimensions of 0.100″ diameter×0.050″ thick. The tray is inserted inside a polyester felt pillow case, while sitting inside a humidity and temperature controlled room or chamber with relative humidity maintain within 45 to 55% and temperature within 20 to 26° C. for optimal self-assembly of the supramolecular layering. After 14 to 24 hours, the pillowcase is removed and the sensors are allowed to further air dry in the same controlled room for an additional 14 to 24 hours. Then the sensor tray is placed inside 40° C. drying oven for 14 to 24 hours. Then the sensor tray is removed and stored inside the humidity and temperature controlled chamber. The sensors are now ready for use or for test.

### Example 3A

### Single Sensing Formulation S66i on Regular-Size SPS for SIR

100 of 0.150″ diameter×0.100″ thick silica porous silicate disks with pore diameter ranging from 200 to 300 angstroms and surface area ranging from 100 to 200 square meter per gram are soaked in a 15 mL of the new S66i sensing reagent mixture containing 7.87 mmole of H₄SiMo₁₂O₄₀.xH₂O, 77.7 mmole of CaCl₂.2H₂O, 2.7 mmole of CCl₃COOH, 0.16 mmole of copper trifluoroacetylacetonate, 1.74 mmole of CuCl₂.2H₂O, 8.6 mmole of CaBr₂2H₂O, 1.126 mmole of Gamma-Cyclodextrin, 0.97 mmole of Hydroxy-Beta-Cyclodextrin, 2.25 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature control room or chamber with relative humidity maintain with 45 to 55% and temperature within 20 to 26° C. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

### Example 3B

### Single Sensing Formulation S66i on Mini-SPS, MICROSIR

7 to 10 microliters of the single sensing element reagent mixture containing 7.87 mmole of H₄SiMo₁₂O₄₀.xH₂O, 77.7 mmole of CaCl₂.H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 2.25 mmole of Na₂PdCl₄, 23.89 mmole of PdCl₂, and 0.55 mmole of Beta-Cyclodextrin is injected directly onto a mini-sized porous silica substrate (SPS) having the dimensions of 0.100″ diameter×0.050″ thick. The impregnated mini-sized substrates are spread flat on a clean Pyrex or plastic tray inside a polyester felt pillow case, while sitting inside a humidity and temperature controlled room or chamber with relative humidity maintain within 45 to 55% and temperature within 20 to 26° C. for optimal self-assembly of the supramolecular layering. After 14 to 24 hours, the pillowcase is removed and the sensors are allowed to further air dry in the same controlled room for an additional 14 to 24 hours. Then the sensor tray is placed inside 40° C. drying oven for 14 to 24 hours. Then the sensor tray is removed and stored inside the humidity and temperature controlled chamber. The sensors are now ready for use or for test.

### Example 4A

### Single Sensing Formulation S66L on Regular-Size SPS for SIR

100 of 0.150″ diameter×0.100″ thick silica porous silicate (SPS) disks with pore diameter ranging from 200 to 300 angstroms and surface area ranging from 100 to 200 square meter per gram are soaked in a 15 mL of the new single sensing element reagent mixture type S66L containing 8.25 mmole of H₄SiMo₁₂O₄₀.xH₂O, 77.7 mmole of CaCl₂.2H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 3.33 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

### Preferred Embodiment 4

### Example 4B

### Single Sensing Formulation S66L on Mini-SPS for MICROSIR M1 and M3

7 to 10 microliters of the single sensing element reagent mixture containing 8.25 mmole of H₄SiMo₁₂O₄₀.xH₂O, 77.7 mmole of CaCl₂.2H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 3.33 mmole of Na₂PdCl₄, 23.89 mmole of PdCl₂, and 0.55 mmole of Beta-Cyclodextrin is injected directly onto a mini-sized porous silica substrate (SPS) having the dimensions of 0.100″ diameter×0.050″ thick. The impregnated mini-sized substrates are spread flat on a clean Pyrex or plastic tray inside a polyester felt pillow case, while sitting inside a humidity and temperature controlled room or chamber with relative humidity maintain within 45 to 55% and temperature within 20 to 26° C. for optimal self-assembly of the supramolecular layering. After 14 to 24 hours, the pillowcase is removed and the sensors are allowed to further air dry in the same controlled room for an additional 14 to 24 hours. Then the sensor tray is placed inside 40° C. drying oven for 14 to 24 hours. Then the sensor tray is removed and stored inside the humidity and temperature controlled chamber. The sensors are now ready for use or for test.

The new formulations for a ONE sensing element system described above have CO detection capability ranges from 30 to 550 ppm. The formulations can be further tuned to have wider ranges of CO detection capabilities simply by increasing the Cu ions concentration from 100 to 1000%. This is necessary for CO alarms to have in order to meet UL 2034 for “Recreational Boats” approval. The current UL 2034 requires CO alarms to detect 6,000 ppm CO within 3 minutes. Since UL also requires that the same CO alarm must also detect as low as 70 ppm CO, “two sensing elements” are needed to cover the full range from 30 to 6,000 ppm CO. Effective Mar. 8, 2007, UL 2034 lowered the upper detection limit to 5,000 ppm for Recreational Boats application.

To differentiate between low and high CO detection range sensors, bromide ions can be removed to give the high-CO-range sensors the yellow appearance, leaving the tan-orange to red appearance for low-CO-range sensors. The yellow high-CO-range sensors are referred to as the “KY” series. Several examples of these formulations with these higher ranges of CO detection capability are shown below.

### Preferred Embodiment 5

### Example 5A

(SIR-02, UL 2034, “Recreational Boats” Applications)

100 of 0.150″ diameter×0.100″ thick silica porous silicate (SPS) disks with pore diameter ranging from 200 to 300 angstroms and surface area ranging from 100 to 200 square meter per gram are soaked in a 15-mL of 7KY type solution, which contains 0.008226391M H₄SiMo₁₂O₄₀, 0.071897966M CaCl₂.2H₂O, 0.014567462M CuCl₂.2H₂O, 0.001069612M Gamma-CD, 0.002013936M Na₂PdCl₄, 0.028761424M PdCl₂, 0.000913589M Beta-CD. After 1 day of soaking, the excess solution is removed and the sensor dried using Kimwipe tissue paper. Sensors are spread flat on a clean Pyrex or plastic tray and allowed to dry slowly inside a polyester felt pillow case inside humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test

### Preferred Embodiment 6

### Example 5B

### Mini-S34 Sensor Series Comprising a Mini-7KY Sensor+a Mini-S6 or Mini-S66 Series in a MICROSIR MOD1-02 (M1-02) or a MOD3-02 (M3-02) for “Recreational Boats” Application per UL 2034

7 to 10 microliters of the 7KY solution containing 0.008226391M H₄SiMo₁₂O₄₀, 0.071897966M CaCl₂.2H₂O, 0.014567462M CuCl₂.2H₂O, 0.001069612M Gamma-CD, 0.002013936M Na₂PdCl₄, 0.028761424M PdCl₂, and 0.000913589M Beta-CD is injected directly onto a mini-sized porous silica substrate (SPS) having the dimensions of 0.100″ diameter×0.050″ thick. The impregnated mini-sized substrates are spread flat on a clean Pyrex or plastic tray inside a polyester felt pillow case, while sitting inside a humidity and temperature controlled room or chamber with relative humidity maintain within 45 to 55% and temperature within 20 to 26° C. for optimal self-assembly of the supramolecular layering. After 14 to 24 hours, the pillowcase is removed and the sensors are allowed to further air dry in the same controlled room for an additional 14 to 24 hours. Then the sensor tray is placed inside 40° C. drying oven for 14 to 24 hours. Then the sensor tray is removed and stored inside the humidity and temperature controlled chamber. The sensors are now ready for use or for test.

Other new formulations to increase to the SENSITIVITY of the chemical sensors to CO after having been stored at very low relative humidity for an extended period of time involved the replacement of CaCl₂ with AlCl₃, CdCl₂, CoCl₂, CeCl₃, CrCl₃, FeCl₃, MnCl₂, NiCl₂, SrCl₂, ZnCl₂, SnCl₂, BaCl₂, MgCl₂, Mg(NO₃)₂, NaBr, NaCl, NaHSO₄, Mg(NO₃)₂, KCO₃, KCl, and/or MgSO₄. The formulations are referred to as the MO37-32 and MO37-64 series. Some of the formulations that yielded positive results are described below.

### Preferred Embodiment 6

### Example 6

### Single CO Sensing SIR, UL Residential and Recreational Vehicle

100 of 0.150″ diameter×0.100″ thick silica porous silicate (SPS) disks with pore diameter ranging from 200 to 300 angstroms and surface area ranging from 100 to 200 square meter per gram are soaked in a 15-mL 0.008226398M H₄SiMo₁₂O₄₀, 0.001069613M Gamma-CD, 0.00091359 M Beta-CD, 0.071898031M MnCl₂.4H₂O, 0.00202082M CuCl₂.2H₂O, 0.002013938M Na₂PdCl₄, and 0.02876145M PdCl₂. After 1 day of soaking, the excess solution is removed and the sensor dried using Kimwipe tissue papers. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

### Preferred Embodiment 7

### Example 7

### Single CO Sensing SIR, UL Residential and Recreational Vehicle

100 of 0.150″ diameter×0.100″ thick silica porous silicate (SPS) disks with pore diameter ranging from 200 to 300 angstroms and surface area ranging from 100 to 200 square meter per gram are soaked in a 15-mL 0.008226398M H₄SiMo₁₂O₄₀, 0.001069613M Gamma-CD, 0.00091359M Beta-CD, 0.071898031M CeCl₃, 0.00202082M CuCl₂.2H₂O, 0.002013938M Na₂PdCl₄, and 0.02876145M PdCl₂. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

Ca replacement by chloride and bromide salts of Sr, Zn, Ni, and Mn has resulted in increase sensitivity to CO at extreme test conditions such as 66° C./40% RH and 61° C./93% RH. It was also observed that different mixture proportions of these salts yield different level of sensitivity gain/loss. One of most desired proportions is detailed in “preferred embodiment 8” below.

### Preferred Embodiment 8

### Example 8B

### Single Sensing Mini-SPS S6e w/ Ca Replaced by Zn to Increase Sensitivity at 66° C./40% RH and 61° C./93% RH

“Soak Method”

600 of the mini-sized silica porous silicate disks with pore diameter ranging from 200 to 300 angstroms and surface area ranging from 100 to 200 square meter per gram are soaked in a 15-mL of the new S6e sensing formulation containing 7.7 mmole of H₄SiMo₁₂O₄₀.xH2O, 38.9 mmole ZnCl₂, 38.9 mmole ZnBr₂, 2.7 mmole of CCl₃COOH, 0.16 mmole of copper trifluoroacetylacetonate, 1.74 mmole of CuCl₂.2H₂O, 8.6 mmole of CaBr2.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside an humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

Another new group of formulations to increase to the SENSITIVITY of the chemical sensors to CO after having been stored at very low relative humidity for an extended period of time involved the addition of AlCl₃, CdCl₂, COCl₂, CeCl₃, CrCl₃, FeCl₃, MnCl₂, NiCl₂, SrCl₂, or ZnCl₂ to the Single Sensing Formulation type S6e as detailed in Example 1. This new group of formulations is known as the MO37-141 series. Additives such as AlCl₃, CdCl₂, CrCl₃, MnCl₂, SrCl₂, and ZnCl₂ were confirmed to have increase SENSITIVITY to CO at low relative humidity conditions.

Table X1

Low % Relative Humidity Long-Term-CO Sensitivity Measurement

MO37-141 Series involves additions of various chlorides of transitional metal to the Single CO sensing formulation S6e. Comparison of Confirmed CO Sensitivities for S6e+Additives and those of S6e without additives and the current dual sensing element S34: Sensors only (no reservoir effect), were stored inside a chamber containing a saturated salt of LiCl for maintaining relative humidity within 11-15% RH at room temperature. After 168 hours, the CO was injected to create 150 ppm. Sensitivity of each sensor was measured at the end of 20 minutes at 150 ppm CO. A sensitivity of 2 represents a 50% change. A single sensing element S6e is more sensitive than the dual sensing elements S34. Additive No. 1, 2, 5, 7, 9, and 10 causes the sensor sensitivity to be greater than those of S6e and S34.

| | QTY. OF | CONFIRMED |
|---|---|---|
| ADDITIVES | SENSING | SENSITIVITY |
| TO S6e | ELEMENT | AT 13 ± 2% RH |
| AlCl₃ | 1 | 3.5 |
| CdCl₂ | 1 | 3.3 |
| COCl₂ | 1 | 2.3 |
| CeCl₃ | 1 | 1.0 |
| CrCl₃ | 1 | 4.9 |
| FeCl₃ | 1 | 1.8 |
| MnCl₂ | 1 | 2.7 |
| NiCl₂ | 1 | 2.2 |
| SrCl₂ | 1 | 2.5 |
| ZnCl₂ | 1 | 3.7 |
| S6e control | 1 | 2.4 |
| S34 current | 2 | 2.1 |

Table X2

CO Sensitivity Measurement at 66° C. and 40% RH Following 30 Days Soaked at 66° C. and 40% RH

Additional confirmed improved performance at 66° C. and 40% RH was found in formulations involving partial to complete replacement CaCl₂ in S6e with MnCl₂ and MnBr₂. Also found was a decreased in sensitivity when a partial to complete CaCl₂ replacement was made with SrCl2 and SrBr2. It was discovered that different proportions of ClCl₂, MnCl₂, MnBr₂, SrCl₂ and/or SrBr₂ yielded different levels of CO sensitivity. Single sensing mini-sized SPS was used in this experiment. They were singly installed in the MICROSIR MOD1-01 assembly configuration (FIG. 7) then mounted on the 8UP-MICROSIR-voltage output board, so the sensor output is converted to a voltage level corresponding to the obscuration of light passing through the MICROSIR CO sensing element. The signal conditioning is performed by a test circuit containing an operational amplifier (OpAmp). The amplification circuit is set to attain an initial value of 4 Volts output. As the sensor responds to CO, the voltage output decreases. This voltage-output board is a subject of a co-pending U.S. Provisional Patent Application No. 60/711,748, filed on Aug. 25, 2005. The complete assembled samples were then stored inside a Thermotron environmental chamber, which maintained at 66° C. % RH and 40% RH for 30 days. At the end of the 30^{th} day, the CO was injected to create 400 ppm. Sensitivity of each sensor was measured for 15 minutes. Change in voltage in response to 400 ppm CO for 15 minutes was calculated and summarized below. Proportion combination #1 is actually the control S6e with change in voltage of less than (<) 0.05 was observed. Two proportion combinations, which have better performances than that of the control, are #3 and 5. All other proportion combination #s are actually worst than the control.

| Proportion | | | | Confirmed Performance |
|---|---|---|---|---|
| Combination # | CaCl₂ | MnCl₂ | MnBr₂ | at 66 C./40% RH |
| 1 | 1 | 0 | 0 | <0.05 |
| 2 | 0 | 1 | 0 | <0.05 |
| 3 | 0 | 0.5 | 0.5 | 0.1 |
| 4 | 0.5 | 0.5 | 0 | 0.05 |
| 5 | 0.5 | 0 | 0.5 | 0.07 |

| Proportion | | | | Confirmed Performance |
|---|---|---|---|---|
| Combination # | CaCl₂ | SrCl₂ | SrBr₂ | at 66° C./40% RH |
| 7 | 0 | 1 | 0 | <0.01 |
| 8 | 0 | 0.5 | 0.5 | <0.01 |
| 9 | 0.5 | 0.5 | 0 | 0.01 |
| 10 | 0.5 | 0 | 0.5 | 0.05 |

Additional tests of the identical samples reported in Table X2 were tested at −40 C, 61° C./93% RH, and 23° C./10% RH. Due to too much electronic noise, the results are not obtainable at −40° C. and 61° C./93% RH. The electronic test board was already ruined in 61 C/93% RH test by the time the samples reached 23° C./10% RH, last test condition of the required UL test “Sequence.” Test boards needed good protective coating for extreme test conditions such as the 61° C./93% RH.

While partial to full replacement of CaCl₂ with MnCl₂, MnBr₂, SrCl₂, and/or SrBr₂ yielded some improved performances at 66° C./40% RH, addition of these same chemicals to S6e formulation does not yield fruitful results in either a −40° C. or a 66° C./40% RH test.

Table X3

CO Sensitivity Measurement at 66° C. and 40% RH Following 30 Days Soaked at 66° C. and 40% RH

Additional confirmed improved performance at 66 C and 40% RH was found in formulations involving partial to complete replacement of CaCl₂ in S6e with ZnCl₂ and ZnBr₂. Also found was a decrease in sensitivity when a partial to complete CaCl₂ replacement was made with NiCl₂ and NiBr₂. It was discovered that different proportions of ClCl₂, ZnCl₂, ZnBr₂, NiCl₂ and/or NiBr₂ yielded different levels of CO sensitivity. Single sensing mini-sized SPS was used in this experiment. They were singly installed in the MICROSIR MOD1-01 assembly configuration (FIG. 7) then mounted on the 8UP-MICROSIR-voltage output board, so the sensor output is converted to a voltage level corresponding to the obscuration of light passing through the MICROSIR CO sensing element. The signal conditioning is performed by a test circuit containing an operational amplifier (OpAmp). The amplification circuit is set to attain an initial value of 4 Volts output. As the sensor responds to CO, the voltage output decreases. This voltage-output board is a subject of a co-pending U.S. Provisional Patent Application No. 60/711,748, filed Aug. 25, 2005. The complete assembled samples were then stored inside a Thermotron environmental chamber, which maintained at 66° C. % RH and 40% RH for 30 days. At the end of the 30^{th} day, the CO was injected to create 400 ppm. Sensitivity of each sensor was measured for 15 minutes. Change in voltage in response to 400 ppm CO for 15 minutes was calculated and summarized below. Proportion combination #1 is actually the control S6e with change in voltage of 0.15V. Note it may seem contradicting when comparing the performance of the control used in this experiment to that of the control S6e used in Table X2. The differences may be caused by a test-to-test variation. For it is important to compare the performances of the experimental sensors to that of the control used in the same given test. Proportion combinations involving ZnCl₂ and ZnBr₂ that yielded better response than the control are C, D, and 9 with the voltage change of 0.3V, 0.2V, and 0.9V, respectively. None of proportion combinations involving NiCl₂ and NiBr₂ yielded any better performances than the control, which had voltage change of 0.15V. Following th is test, the samples were tested at −40° C. then 61° C./93% RHC, which are detailed below.

| | | | | Confirmed Performance |
|---|---|---|---|---|
| Proportion | | | | at 66° C./40% RH |
| Combination # | CaCl₂ | ZnCl₂ | ZnBr₂ | Change in Voltage (Volt) |
| A | 1 | 0 | 0 | 0.15 (Control) |
| B | 0 | 1 | 0 | 0.04 |
| C | 0 | 0.5 | 0.5 | 0.3 |
| D | 0.5 | 0.5 | 0 | 0.2 |
| E | 0.5 | 0 | 0.5 | 0.9 |

| | | | | Confirmed Performance |
|---|---|---|---|---|
| Proportion | | | | at 66° C./40% RH |
| Combination # | CaCl₂ | NiCl₂ | NiBr₂ | Change in Voltage (Volt) |
| F | 0 | 1 | 0 | 0.03 |
| G | 0 | 0.5 | 0.5 | 0.06 |
| H | 0.5 | 0.5 | 0 | 0.05 |
| I | 0.5 | 0 | 0.5 | 0.07 |

Like those samples reported in Table X2, these samples were also tested at −40° C. Again, due to too much electronic noise, the results were not obtainable at −40° C. The samples should be retested using a more electronically stable test board.

When tested at 61° C./93% RH, there was also electronic noise that some of the test sites on the 8up voltage-output boards were not able to generate meaningful results. But some sites were in adequate condition enough to capture certain performances of certain sensor formulations, which are summarized in Table X4 below.

Table X4

CO Sensitivity Measurement at 61° C. and 93% RH Following 10 Days Soaked at 61° C. and 93% RH

Test results of the exact same samples reported in Table X3 at 61° C./93% RH following the 66° C./40% RH and the −40° C. (which was invalid due to noise). The samples were preconditioned inside the Thermotron environmental chamber at 61° C./93% RH for 10 days. At the end of the 10^{th} day, CO was injected into the chamber to create and to maintain within 400±10 ppm CO for 15 minutes. Change in voltage in response to 400 ppm CO for 15 minutes was calculated and summarized below. Fortunately, there was a valid result for the control of 0.05V to be used as a benchmark for comparison. According to data, all obtainable results for proportion combinations C, G, H, and I are at least 4 times more sensitive than the control. Results were not obtainable (?) for proportion combinations B, D, E, and F. They should be re-tested using a more robust electronic test boards.

| | | | | Confirmed Performance |
|---|---|---|---|---|
| Proportion | | | | at 61° C./93% RH |
| Combination # | CaCl₂ | ZnCl₂ | ZnBr₂ | Change in Voltage (Volt) |
| A | 1 | 0 | 0 | 0.05 (Control) |
| B | 0 | 1 | 0 | ? |
| C | 0 | 0.5 | 0.5 | 0.3 |
| D | 0.5 | 0.5 | 0 | ? |
| E | 0.5 | 0 | 0.5 | ? |

| | | | | Confirmed Performance |
|---|---|---|---|---|
| Proportion | | | | at 61° C./93% RH |
| Combination # | CaCl₂ | NiCl₂ | NiBr₂ | Change in Voltage (Volt) |
| F | 0 | 1 | 0 | ? |
| G | 0 | 0.5 | 0.5 | 0.3 |
| H | 0.5 | 0.5 | 0 | 0.2 |
| I | 0.5 | 0 | 0.5 | 0.3 |

Based on the obtainable results shown in Tables X3 and X4, proportion combination C appears to be the best among all other combinations because it is two times more sensitive than the control at 66° C./40% RH and six times better than the control at 61° C./93% RH.

Table Y

High % Relative Humidity Long-Term-CO Sensitivity Measurement

Based on the confirmed CO sensitivity of the MO37-141 and MO37-34 Series, it is predicted that a combination of bromide and chloride salts of the same transitional metal would results in increase CO sensitivity after the sensors have been stored at both LOW and HIGH relative humidity conditions for an extended period of time.

Predicted CO Sensitivity for the following: S6e with the additions of Bromide and Chloride salts of transitional metals, S6e with Bromide and Chloride salts of Ca and Cu replaced by Bromide and Chloride salts of transitional metals,

| | | CONFIRMED CO | PREDICTED CO |
|---|---|---|---|
| | # OF SENSING | SENSITIVITY AT | SENSITIVITY AT |
| ADDITIVES TO S6e | ELEMENT | 13 ± 2% RH | 95 ± 4% RH |
| 1. AlCl₃ &AlBr₃ | 1 | + | + |
| 2. CdCl₂&CdBr₂ | 1 | + | + |
| 3. COCl₂&Co Br₂ | 1 | − | − |
| 4. CeCl₃&CeBr₃ | 1 | − | − |
| 5. CrCl₃&CrBr₃ | 1 | ++ | ++ |
| 6. FeCl₃&FeBr₃ | 1 | − | − |
| 7. MnCl₂&MnBr₂ | 1 | + | + |
| 8. NiCl₂&NiBr₂ | 1 | − | − |
| 9. SrCl₂&ZnBr₂ | 1 | + | + |
| 10. ZnCl₂ & ZnBr₂ | 1 | + | + |
| S6e control | 1 | S6e control | S6e control |
| S34 current | 2 | S34 control | S34 control |

Based on the fact that bromide and chloride salts of certain transitional metal made the sensing element much TOO SENSITIVE at 0° C., it is also suggested that any mixture combinations of these salts might also INCREASE SENSITIVITY to CO at the extreme temperature conditions.

Table Z

Minus (−) 40° C. and +70° C.: CO Sensitivity Testing

Predicted Increased CO Sensitivity at extreme temperature of −40° C. and +70° C.+=INCREASE in SENSITIVITY by having Bromide and Chloride Salts of Transitional metal in the S6e or S66 or the KY sensing formulations.

=DECREASE in SENSITIVITY by having Bromide and Chloride Salts of Transitional metal in the S6e or S66 or the KY sensing formulations.

Bromide and Chloride Salts of Transitional metal # of Sensing Element Predicted CO Sensitivity at minus (−) 40° C. Predicted CO Sensitivity at +70° C.

| BROMIDE AND | | | |
|---|---|---|---|
| CHLORIDE SALTS OF | # OF SENSING | PREDICTED CO | PREDICTED CO |
| TRANSITIONAL METAL | ELEMENT | SENSITVITY AT MINUS (−)40° C. | SENSITIVITY AT +70° C. |
| 1. AlCl₃&AlBr₃ | 1 | + | + |
| 2. CdCl₂&CdBr₂ | 1 | + | + |
| 3. COCl₂ & CoBr₂ | 1 | − | − |
| 4. CeCl₃ & CeBr₃ | 1 | + | + |
| 5. CrCl₃ & CrBr₃ | 1 | + | + |
| 6. FeCl₃ & FeBr₃ | 1 | − | − |
| 7. MnCl₂ & MnBr₂ | 1 | + | + |
| 8. NiCl₂ & NiBr₂ | 1 | − | − |
| 9. SrCl₂ & SrBr₂ | 1 | + | + |
| 10. ZnCl₂ & ZnBr₂ | 1 | + | + |

### Preferred Embodiment 9

### Example 9

### SIR-01, Single CO Sensing Element, UL 2034 Residential and RV

100 of 0.150″ diameter×0.100″ thick silica porous silicate disks with pore diameter ranging from 200 to 300 angstroms and surface area ranging from 100 to 200 square meter per gram are soaked in a 15 mL of the new S6e sensing formulation containing 7.7 mmole of H₄SiMo₁₂O₄₀.xH₂O, 77.7 mmole of CaCl₂.2H₂O, 35.5 mmole MnCl₂.4H₂O, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

### Example 9

### SIR-01, S6e Single CO Sensing Element, UL 2034 Residential and RV

100 of 0.150″ diameter×0.100″ thick silica porous silicate disks with pore diameter ranging from 200 to 300 angstroms and surface area ranging from 100 to 200 square meter per gram are soaked in a 15 mL of the new S6e sensing formulation containing 7.7 mmole of H₄SiMO₁₂O₄₀.xH₂O, 77.7 mmole of CaCl₂.2H₂O, 35.95 mmole CdCl₂, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

### Example 10

One preferred embodiment for dry application such 7-10% RH is shown below in example 10.

(SIR-01, S6e Single CO Sensing Element, UL 2034 Residential and RV)

100 of 0.150″ diameter×0.100″ thick silica porous silicate disks with pore diameter ranging from 200 to 300 angstroms and surface area ranging from 100 to 200 square meter per gram are soaked in a 15 mL of the new S6e sensing formulation containing 7.7 mmole of H₄SiMO₁₂O₄₀.xH₂O, 77.7 mmole of CaCl₂.2H₂O, 35.95 mmole CrCl₃, 2.7 mmole of CCl₃COOH, 0.16 mmole of copper trifluoroacetylacetonate, 1.74 mmole of CuCl₂.2H₂O, 8.6 mmole of CaBr₂.2H₂O, 1.126 mmole of Gamma-Cyclodextrin, 0.97 mmole of Hydroxy-Beta-Cyclodextrin, 1.89 mmole of Na₂PdCl₄, 23.89 mmole of PdCl₂, and 0.55 mmole of Beta-Cyclodextrin. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

### Preferred Embodiment 10

### Example 11

One preferred embodiment for detecting 5 to 10 ppm CO is shown below in example 11. (MICROSIR Models M1-01e, M1-02e, M3-01e, and M3-02e with S50 Single CO Sensing Element, an aid for early fire detection and elimination of false alarm).

“Soak Method”

600 of the mini-sized silica porous silicate disks with pore diameter ranging from 200 to 300 angstroms and surface area ranging from 100 to 200 square meter per gram are soaked in a 15-mL of the new S50 sensing formulation containing 0.01233965M H₄SiMo₁₂O₄₀, 0.001069613M Gamma-Cyclodextrin, 0.00091359 Beta-Cyclodextrin, 0.071898031M CaCl₂.2H₂O, 0.00202082M CuCl₂.2H₂O, 0.018073268M Na₂PdCl₄, and 0.02876145M PdCl₂. After 1 day of soaking, the excess solution is removed and the sensor dried using Kimwipe tissue paper. Sensors are spread flat on a clean Pyrex or plastic tray and allowed to dry slowly inside a polyester felt pillow case inside a humidity and temperature controlled room or chamber with relative humidity maintain within 45 to 55% and temperature within 20 to 26° C. After 1 day, the pillowcase is removed and the sensors are allowed to further air dry in the same controlled room for 1 more day. Then the sensor tray is placed inside 40° C. drying oven for 1 to 2 days. The sensor tray is removed and stored inside the humidity and temperature controlled chamber. The sensors are now ready for use or for test.

The new Single-Chemical-Sensing Element detects CO without any power. It functions adequately, by itself, without a reservoir, as a visual indicator for CO in real-world conditions.

However, like the current Dual-Chemical-Sensing-Elements, the new Single-Chemical-Sensing Element the reservoir is preferred for certain application such as to meet the stringent requirement in UL 2034 and 2075 as well as CSA6.19-01. Some of are UL test requirements are not real world related such as those described in CRITERION 9 below.

The reservoir, according to a co-pending U.S. patent application titled, “Chemical System for Controlling Relative Humidity and Air Quality,” U.S. patent application Ser. No. 10/997,646, filed Nov. 24, 2004, and U.S. Pat. No. 6,251,344 contains a chemical mixture for controlling relative humidity within a specified space.

In these patents, Goldstein, et. al. describe a means to maintain relative humidity and certain air quality contaminates within a predetermined range for a predetermined period of time within a chamber, which is connected to the atmosphere. The objective is to maintain a specific air quality including relative humidity (RH) within a predetermined range for extended period of time under real-world conditions as well as extreme conditions. The controlled chamber(s) is contained within a housing that has one or more small openings to the atmosphere. The relative humidity control system also comprises at least one opening to a reservoir of chemicals including a salt with water in at least some solid or a solution containing at least some excess solid phase salt. This control system maintains predetermined RH % range within the “Controlled Chamber” for a given temperature range regardless of the humidity variations in the outside environment, even allowing operation in a condensing environments. Either the solid or saturated salts in the reservoir can be isolated from the controlled chamber by means of a hydrophobic membrane. These membranes may include, but not limited to, UPE (a polyethylene membrane manufactured by Millipore of Bedford, Mass.) or Goretex (a Teflon membrane manufacturer by W. L. Gore & Associates, Inc.).

These membranes allow water to pass in the gaseous state but not liquid solution or solid. The membrane allows the system to be orientation in any direction, i.e., to be placed in any orientation even with the membrane facing down.

In addition, a getter system is provided which can remove specific airborne contaminants, pollutants, and or warfare agents. The getter can keep items such as chemical sensors to be protected in the controlled environmental chamber, free from contamination and in a specified RH range thus increase its operating life and effectiveness.

Previously, the Dual-CO-Sensing-Elements were used in conjunction with a reservoir system, which contains a mixture of Mg(NO₃)₂.6H₂O and MgSO₄.7H₂O. While this mixture enables the Dual-CO-Sensing-Elements to pass the UL 2034 “Sequential Tests,” from start to finish, it is unable to successfully allow the new Single-CO-Sensing Element to pass the same sequential testing. The new Single-CO-Sensing Element needs a new reservoir system in order to meet the UL 2034 requirement. The reservoir system is detailed in a co-pending U.S. patent application Ser. No. 10/997,646, filed Nov. 24, 2004. The new reservoir contains salt of MnCl₂ instead of Mg(NO₃)₂.6H₂O and MgSO₄.7H₂O.

The following criteria were taken from UL 2034, 2nd. Edition, effective Oct. 1, 1998. Criteria 1 through 10 must be carried out in the extract order. In order for either the dual or the single CO sensing system to pass, it must be able to pass all four different gas concentrations within the allowed lower and upper time limits at the test conditions as specified by UL's SEQUENTIALLY from Criterion 1 to Criterion 11 without having to replace a sensor component.

Table 1

CO concentrations Vs. Time Limits as Specified Under UL 2034

Four CO concentrations along with the upper and lower time limits and acceptance criteria, which applies to all 12 criteria below.

| | LOWER TIME | | UPPER TIME | | |
|---|---|---|---|---|---|
| CO ppm | LIMITS | | LIMITS | | ACCEPTANCE CRITERIA |
| 30 | 8 | hr. | 8 | hr. | Must not alarm for 8 hours. |
| 70 | 60 | min. | 240 | min. | Must not alarm before 60 minutes |
| | | | | | and must alarm by 240 minutes. |
| 150 | 10 | min. | 50 | min. | Must not alarm before 10 minutes |
| | | | | | and must alarm by 50 minutes. |
| 400 | 4 | min. | 15 | min. | Must not alarm before 4 minutes |
| | | | | | and must alarm by 15 minutes. |

Criterion 1: Sensitivity Tests

Preconditioning test samples for 48 hours in a controlled test chamber of about 20-26° C. and about 30-70% RH. After 48 hours, expose the samples to the following CO concentrations. First expose the samples to 70 ppm CO for 240 minutes, then regenerate the samples in air for 2 to 4 hours. Second, expose the samples to 150 ppm CO for 50 minutes, next regenerate the samples in air for 4 to 6 hours. Third, expose the samples to 400 ppm CO for 15 minutes, then regenerate the samples in air for 8 to 16 hours. Finally, expose the samples to 30 ppm CO for 8 hours.

Criterion 2: Stability Test

The exact same samples from criterion 1 are placed inside an environmental chamber (Thermotron), which is programmed to ramp temperature and percent relative humidity cycling from 23° C. and 55% to 0° C. and 15% RH in 15 minutes and hold at 0° C. and 15% RH for 30 minutes, then ramp up to 49° C. and 15% RH in 15 minutes and hold at 49° C. and 15% RH for 15 minutes. The samples must resist false alarming throughout all 10 cycles between 0° C. and 49° C.

Criterion 3: Sensitivity Test Post Stability Test

The samples from Criteria 2 are preconditioned test for 16-24 hours in a controlled test chamber of about 20-26° C. and about 30-70% RH. Then, first expose the samples to 70 ppm CO for 240 minutes, then regenerate the samples in air for 2 to 4 hours. Second, expose the samples to 150 ppm CO for 50 minutes, then regenerate the samples in air for 4 to 6 hours. Third, expose the samples to 400 ppm CO for 15 minutes, then regenerate the samples in air for 8 to 16 hours. Fourth

Criterion 4: Selectivity Test

Inside a test chamber at 20-26° C. and 30-70% RH, the same samples from criterion 3 are to be exposed for 2 hours in each of the following gases with approximately 1 hour of regeneration time in air between gases: 500 ppm methane, 300 ppm butane, 500 ppm Heptane, 200 ppm ethyl acetate, 200 ppm isopropanol, and 5,000 ppm carbon dioxide. Samples must resist false alarming to all of the 6 gases.

Criterion 5: Sensitivity Test Post Selectivity Test

The same samples from Criteria 4 are preconditioned for 16-24 hours in a controlled test chamber of about 20-26° C. and about 30-70% RH. Then, first expose the samples to 70 ppm CO for 240 minutes, then regenerate the samples in air for 2 to 4 hours. Second, expose the samples to 150 ppm CO for 50 minutes, then regenerate the samples in air for 4 to 6 hours. Third, expose the samples to 400 ppm CO for 15 minutes, then regenerate the samples in air for 8 to 16 hours. Fourth, expose the samples to 30 ppm CO for 8 hours.

Criterion 6: Sensitivity Test During 49° C. and 40% RH

The same samples from Criteria 5 are preconditioned for 3 hours in a controlled test chamber of about 49° C. and about 40% RH. Then, first expose the samples to 70 ppm CO for 240 minutes, then regenerate the samples in air for 2 to 4 hours. Second, expose the samples to 150 ppm CO for 50 minutes, then regenerate the samples in air for 4 to 6 hours. Third, expose the samples to 400 ppm CO for 15 minutes, then regenerate the samples in air for 8 to 16 hours. Fourth, expose the samples to 30 ppm CO for 8 hours.

Criterion 7: Sensitivity Test During 0° C. and 15% RH

The same samples from Criteria 6 are preconditioned for 3 hours in a controlled test chamber of about 0° C. and about 15% RH. Then, first expose the samples to 70 ppm CO for 240 minutes, then regenerate the samples in air for 2 to 4 hours. Second, expose the samples to 150 ppm CO for 50 minutes, then regenerate the samples in air for 4 to 6 hours. Third, expose the samples to 400 ppm CO for 15 minutes, then regenerate the samples in air for 8 to 16 hours. Fourth, expose the samples to 30 ppm CO for 8 hours.

Criteria 8: Shipping and Storage Test

The exact same samples from criterion 7 are placed inside an environmental chamber, which is programmed to ramp temperature and percent relative humidity cycling from 23° C. and 55% to 70° C. and 40% RH in 3 hours and hold at 70° C. and 40% RH for 24 hours, then ramp down to minus (−) 40° C. and 15% RH in 3 hours and hold at minus (−)40° C. and 15% RH for 3 hours. The samples must resist false alarming throughout the test duration.

Criterion 9: Sensitivity Test Post Shipping & Storage Test

The same samples from Criteria 8 are preconditioned for 16-24 hours in a controlled test chamber of about 20-26° C. and about 30-70% RH. Then, first expose the samples to 70 ppm CO for 240 minutes, then regenerate the samples in air for 2 to 4 hours. Second, expose the samples to 150 ppm CO for 50 minutes, then regenerate the samples in air for 4 to 6 hours. Third, expose the samples to 400 ppm CO for 15 minutes, then regenerate the samples in air for 8 to 16 hours. Fourth, expose the samples to 30 ppm CO for 8 hours.

Criteria 10: Sensitivity Test During 52° C. and 95% RH

The same samples from Criteria 9 are preconditioned for 168 hours at 52° C. and 95% RH in an environmental chamber. After samples resist false alarming for 168 hours, they are to be exposed to the following CO concentrations. First expose the samples to 70 ppm CO for 240 minutes, then regenerate the samples in air for 16 to 24 hours. Second, expose the samples to 150 ppm CO for 50 minutes, then regenerate the samples in air for 16 to 24 hours. Third, expose the samples to 400 ppm CO for 15 minutes, then regenerate the samples in air for 24 to 48 hours. Fourth, expose the samples to 30 ppm CO for 8 hours.

Criteria 11: Sensitivity Test During 23° C. and 15% RH

The same samples from Criteria 10 are preconditioned for 168 hours at 23° C. and 15% RH in an environmental chamber. After samples resist false alarming for 168 hours, they are to be exposed to the following CO concentrations. First expose the samples to 70 ppm CO for 240 minutes, then regenerate the samples in air for 16 to 24 hours. Second, expose the samples to 150 ppm CO for 50 minutes, then regenerate the samples in air for 16 to 24 hours. Third, expose the samples to 400 ppm CO for 15 minutes, then regenerate the samples in air for 16 to 24 hours. Fourth, expose the samples to 30 ppm CO for 8 hours.

Criteria 12A: Sensitivity Test During 22±3° C. and 10±3% RH

This new UL 2034 requirement went into effect on Mar. 8, 2007. It will replace the current criterion 11 above. Like the current criteria 11, the same samples from Criteria 10 are preconditioned for 168 hours at 23° C. and 10±3% RH in an environmental chamber. After samples resist false alarming for 168 hours, they are to be exposed to the following CO concentrations. First expose the samples to 70 ppm CO for 240 minutes, then regenerate the samples in air for 16 to 24 hours. Second, expose the samples to 150 ppm CO for 50 minutes, then regenerate the samples in air for 16 to 24 hours. Third, expose the samples to 400 ppm CO for 15 minutes, then regenerate the samples in air for 16 to 24 hours. Fourth, expose the samples to 30 ppm CO for 8 hours.

The present invention is useful for the detection of carbon monoxide from fires, automobiles, appliances, motors, and other sources. Unlike the DUAL CO sensing system disclosed in U.S. Pat. No. 5,618,493, only a SINGLE sensing element is needed to meet UL 2034 or CSA 6.19-01 requirements for residential and recreational vehicle applications. The single sensing element also has long functional life of at least 6 years. It costs less than the dual sensing system; and is also very CO specific. In addition, it is also self-calibrated. The comparative data, which verify these statements, are shown in Tables 2 and 3.

Criteria 12B: Sensitivity Test During 22±3° C. and 7.5±5% RH

Similar to Criterion 12A for UL 2034, UL 2075 requires for low humidity to 7.5±0.5% RH at 22±3° C. It replaced the current criteria 11 above, effective Mar. 8, 2007. UL 2075 applies to CO alarms used in “system detection” application. Like the current criteria 11, the same samples from Criteria 10 are preconditioned for 168 hours at 22° C. and 7.5±0.5% RH in an environmental chamber. After samples resist false alarming for 168 hours, they are to be exposed to the following CO concentrations. First expose the samples to 70 ppm CO for 240 minutes, then regenerate the samples in air for no more than 16 hours. Second, expose the samples to 150 ppm CO for 50 minutes, then regenerate the samples in air for not more than 16 hours. Third, expose the samples to 400 ppm CO for 15 minutes, then regenerate the samples in air for not more than 16 hours. Fourth, expose the samples to 30 ppm CO for 8 hours.

Table 2

Various NEW mini-sized “Single CO Sensing Elements”, manufactured according to examples 1B, 2B, 3B, and 4B, were compared against the current regular-sized “Dual CO Sensing Elements.” All samples are assembled with reservoirs containing MnCl2. All samples were on 9SIR-MICROSIR PCB boards. Comparison was based on Criteria 1, 6, and 7. “+” indicate “great than or equal to 90% passing rate” for all of the CO concentrations and time limits shown in Table 1. “−” Indicates “below 90% passing rate.”

| SENSOR | DISK | APPLICABLE | CRITERION 1 | CRITERION 6 | CRITERION 7 |
|---|---|---|---|---|---|
| TYPE | QTY | EXAMPLE | 55% RH/23° C. | 40%/RH/49° C. | 15%/RH/0° C. |
| S6e | 1 | 1B | + | + | + |
| S66e | 1 | 2B | + | + | + |
| S66i | 1 | 3B | + | + | + |
| S66L | 1 | 4B | + | + | + |
| S34 | 2 | Current S34 | + | + | + |

Table 3

Various NEW mini-sized “Single CO Sensing Elements”, manufactured according to examples 1B, 2B, 3B, and 4B, were compared against the current regular-sized “Dual CO Sensing Elements.” All samples are assembled with reservoirs containing MnCl₂. All samples were on 9SIR-MICROSIR PCB boards. Comparison was based on Criteria 10, 11, and 12. “+” Indicates “great than or equal to 90% passing rate” for all of the CO concentrations and time limits shown in Table 1. “−” Indicates “below 90% passing rate.”

| SENSOR | DISK | APPLICABLE | CRITERION 10 | CRITERION 11 | CRITERION 12 |
|---|---|---|---|---|---|
| TYPE | QTY | EXAMPLE | 95% RH/52° C. | 15% RH/23° C. | 10% RH/23° C. |
| S6e | 1 | 1B | + | + | + |
| S66e | 1 | 2B | + | + | + |
| S66i | 1 | 3B | + | + | + |
| S66L | 1 | 4B | + | + | + |
| S34 | 2 | Current S34 | + | + | + |

### Preferred Embodiments Versus Applications

Example 2B is highly preferred in MICROSIR application for meeting UL 2034 or CSA 6.19-01 residential application. This is single sensing element formulation S66e.

Example 4A is best for SIR application for meeting UL 2034 or CSA 6.19-01 residential application. This is the single sensing element formulation S66L.

Example 4A+Example 5A combined, are highly preferred for SIR application for meeting UL 2034 for “Recreational Boating,” application.

Example 4B is preferred for MICROSIR application to meet UL 2034 or CSA 6.19-01 for “Recreational Vehicle” application.

Examples 1A, 2A, or 4A, is best for CO Visual Indicator application. Below is the confirmed, comparative performances of QuantumEye's 34t and S6e performance. The sensitivity of S66e, S66L, and S66i are greater than that of S6e.

The SECOND application for the NEW “Single CO Sensing Element,” is LOW COST VISUAL INDICATOR for CO. It is preferred that the regular-sized disks are used for this applicable for better visual effect. As stated above, the new Single CO Sensing Element functions as a VISUAL COLOR indicator for WARNING the end users the presence of CO. It provides the LOW-COST alternative for protecting human life against the danger of CO poisoning.

Currently, there are three (3) different visual color indicators for CO commercially available. First is the “QUANTUM EYE”, which is manufactured according to U.S. Pat. No. 5,063,164, by QUANTUM GROUP INC. located in San Diego, Calif. Second is the “DEAD STOP,” which manufactured in Denmark for J L SIMS COMPANY, INC. located in St. Louis, Mo. Third is “AIR ZONE,” which is supplied by ENZONE Inc. located in Davie, Fla.

Currently, “Quantum Eyes” are made with sol-gel substrates, which are manufactured by GEL-TECH in Orlando, Fla. These substrates are very costly due to low manufacturing yields, which results from poor mechanical strength. The present invention provides low cost visual CO detectors called the “S6e” sensor series, which are mechanically strong. Initially, S6e appears tan-orange and turns dark blue upon exposure to danger levels of CO, i.e. 70 ppm and above. S6e QuantumEye™ returns to its initial color after CO is removed. S6e Quantum Eyes fail-safe as they will become more and more sensitivity towards CO after have repeatedly re-exposed to CO 50 to 100 times.

While there are no regulatory standards that govern visual CO indicators, the new S6e Quantum-Eye out-performed DEAD STOP and AIR ZONE under a wider range of temperature and relative humidity such as −40° C. to +70° C. and 25 to 95% RH as well as meeting the OSHA limits by NOT changing colors at 50 ppm CO for 8 hours. Changing COLOR in response to 50-ppm CO is considered to be “false-alarming”. The new “S6e Quantum-Eye” has long functional life and is self-regenerated. It is cost effective and is very CO specific.

The comparative data, which verifies these statements, are shown in Tables 4-7 below.

According to the Coburn's equation for determining the effect of CO poisoning in human at different levels of percentage carboxyhemoglobin (% COHb) in the blood, the exposure to 200 ppm CO at various exposure times yields the following symptoms: 1) 35 minutes equals 10% COHb (no effect), 2) 60 minutes equals 15% COHb (slight headache), and 90 minutes equals 20% COHb (Headache).

For CO Sensitivity Test, a visual CO indicator must indicate CAUTION within 60 minutes and DANGER within 90 minutes when exposed to 200 ppm CO to be considered “pass” or “+”. Any visual CO indicator that cannot meet these criteria would be considered “fail” or “−”.

For Resistance to Low CO Concentration Test, a visual CO indicator must NOT change color to indicator neither CAUTION nor DANGER when exposed to 50 ppm CO for 8 hours.

Table 4

CO Sensitivity Test Comparison at Variable Ambient Relative Humidity and Room Temperature Test

Two visual CO indicators each were randomly chosen from the groups of the new S6e Quantum Eyes, 34t Quantum Eyes, Dead-Stop, and Air-Zone. They were placed inside test chamber and allowed to equilibrate for 24 to 48 hours at each test condition before they were exposed to 200 ppm CO for 90 minutes. Each unit must indicate “CAUTION” within 60 minutes to be considered pass (+) and a “DANGER” within 90 minutes to be considered pass (+) at each test conditions. Unit that cannot failed to meet these time limits were considered to be failing (−). S6e Quantum Eyes and 34t Quantum Eyes are the only two groups that could pass all three-test conditions.

| | 20° C. & 15% RH | | 20° C. & 53% RH | | 20° C. & 90% RH | |
|---|---|---|---|---|---|---|
| | 24 Hr. | | 24 Hr. | | 48 Hr. | |
| Samples | CAUTION | DANGER | CAUTION | DANGER | CAUTION | DANGER |
| S6e Quantum Eyes | + | + | + | + | + | + |
| 34t Quantum Eyes | + | + | + | + | + | + |
| Dead-Stop | − | − | + | + | + | + |
| Air-Zone | − | − | + | + | + | + |

Table 5

Comparison of Low CO Level Resistance—Variable Ambient Relative Humidity/Room Temperature Test

Two visual CO indicators each of the Model types She Quantum Eyes, 34t Quantum Eyes, Dead-Stop, and Air-Zone were placed inside a test chamber and allowed to equilibrate for 15 to 20 minutes at each test condition. Then, they were exposed to 50-ppm CO for 8 hours. “+”=unit that passed in NOT indicating the 50 ppm-CO for the entire 8 hours. “−”=unit that failed because they indicate the presence of 50-ppm CO when they were not supposed to. Only S6e Quantum Eyes and Air-Zone pass this test. However, Air-Zone had already failed the sensitivity comparison test as described in Table 4.

| | 20° C. & | 20° C. & | 20° C. & |
|---|---|---|---|
| TEST CONDITION | 33% RH | 53% RH | 67% RH |
| S6e Quantum Eyes | + | + | + |
| 34t Quantum Eyes | − | − | − |
| Dead-Stop | − | − | − |
| Air-Zone | + | + | + |

Table 6

Comparative of Sensitivities-Variable Ambient Relative Humidity/High Temperature Test

Two visual CO indicators each were randomly chosen from the groups of S6e Quantum Eyes, 34t Quantum Eyes, Dead-Stop, and Air-Zone. They were placed inside a Thermotron environmental test chamber and allowed to equilibrate for 1 to 48 hours as specified in the Table below. While at each test condition, they were exposed to 200 ppm CO was for 90 minutes. “+”=unit that indicated the “CAUTION” within 60 minutes and/or “DANGER” within 90 minutes. “−”=unit that failed to indicate “CAUTION” within 60 minutes and/or “DANGER” within 90 minutes. S6e Quantum Eyes and 34t Quantum Eyes are the only two groups that met this requirement.

| | 40° C. & 40% RH | | 70° C. & 40% RH | | 50° C. & 95% RH | |
|---|---|---|---|---|---|---|
| | 3 Hr. Conditioning | | 1 Hr. Conditioning. | | 48 Hr. Conditioning | |
| Samples | CAUTION | DANGER | CAUTION | DANGER | CAUTION | DANGER |
| S6e Quantum Eyes | + | + | + | + | + | + |
| 34t Quantum Eyes | + | + | + | + | + | + |
| Dead-Stop | − | − | − | − | + | + |
| Air-Zone | − | − | − | − | + | + |

Table 7

Comparison of CO Sensitivity-Low Relative Humidity/Low Temperature Test

Two visual CO indicators each were randomly chosen from the groups of S6e Quantum Eyes, 34t Quantum Eyes, Dead-Stop, and Air-Zone. They were placed inside a Thermotron environmental test chamber and allowed to equilibrate to each test condition for 3 hours. While at each test condition, they were exposed to 200 ppm CO was for 90 minutes. “+”=unit that indicated “CAUTION” within 60 minutes and/or “DANGER” within 90 minutes. “−”=unit that failed to indicate “CAUTION” within 60 minutes and/or “DANGER” within 90 minutes. S6e Quantum Eyes and 34t Quantum Eyes are the only two groups that met this requirement.

| | 0° C. & 15% RH | | Minus (−) 40° C. | |
|---|---|---|---|---|
| Sample | 3 Hr. | | 3 Hr. | |
| Descriptions | CAUTION | DANGER | CAUTION | DANGER |
| S6e Quantum Eyes | + | + | + | + |
| 34t Quantum Eyes | + | + | + | + |
| Dead-Stop | − | − | − | − |
| Air-Zone | − | − | − | − |

The THIRD application for the NEW “Single CO Sensing Element,” is DIGITAL CO ALARMS. When the NEW mini-sized S6 and S66 series were enclosed in the MICROSIR reservoirs assembly and then tested on yet another newly invented printed circuit board, which is a subject of another co-pending patent application titled, “Digital Gas Detector and Noise Reduction Techniques”, U.S. patent application No. which Gonzales describes a set of equations that convert and correlate the NEW sensor responses to CO ppm on LCD display. The results from the first prototype digital CO alarm using a single CO sensing element were encouraging and are shown in Tables 8 and 9.

Table 8

Comparison of LCD Display in Terms of PPM CO-Ambient Relative Humidity/Ambient Temperature Test

Quantum's prototype MICROSIR digital CO alarm versus display the actual CO concentration as indicated by a Dräger Pac-III (electrochemical based sensor, manufactured Dräger Inc., can be purchased for about $3,000 US dollars). Quantum's prototype MICROSIR digital CO alarm comprised Quantum's new “single CO sensing element” type S66L on the new electronic board and software. According to UL 2034, the limits for 70-ppm CO are from 60 to 240 minutes. It is highly preferred that an LCD display does NOT show any CO concentration for the first 59 minutes at this concentration to prevent premature WARNING to the end users; hence, reducing false alarm. Therefore, the fact that MICROSIR CO alarm did not display CO concentration for first 6 minutes is actually a good thing. The accuracy of the prototype MICROSIR digital CO alarm was within ±13% in 70 ppm CO, after 9 minutes without any calibration.

Table 8 Continued

Comparison of LCD Display in Terms of PPM CO-Ambient Relative Humidity/Ambient Temperature Test

| | | Prototype MICROSIR |
|---|---|---|
| Elapsed | Reference CO | Digital CO Alarm With |
| Time (Min.) | Conc. (ppm) | S66L Single Sensing Element |
| 0 | 0 | 0 |
| 1 | 69 | 0 |
| 2 | 71 | 0 |
| 3 | 71 | 0 |
| 4 | 71 | 0 |
| 5 | 71 | 0 |
| 6 | 71 | 0 |
| 7 | 71 | 0 |
| 8 | 70 | 70 |
| 9 | 70 | 70 |
| 10 | 71 | 75 |
| 11 | 70 | 75 |
| 12 | 70 | 77 |
| 13 | 70 | 77 |
| 14 | 70 | 79 |
| 15 | 70 | 79 |
| 16 | 70 | 79 |
| 17 | 70 | 79 |
| 18 | 70 | 78 |
| 19 | 70 | 78 |
| 20 | 70 | 74 |
| 21 | 70 | 74 |
| 22 | 69 | 74 |
| 23 | 69 | 74 |
| 24 | 71 | 71 |
| 25 | 70 | 71 |
| 26 | 70 | 70 |
| 27 | 70 | 70 |

Table 9

Comparison of LCD Display in Terms of PPM CO-Ambient Relative Humidity/Ambient Temperature Test

Quantum's prototype MICROSIR digital CO alarm versus display the actual CO concentration as indicated by a Dräger Pac-III (electrochemical based sensor, manufactured Dräger Inc., can be purchased for about $3,000 US dollars). Quantum's prototype MICROSIR digital CO alarm comprised Quantum's new “single CO sensing element” type S66L on the new electronic board and software. According to UL 2034, the limits for 150-ppm CO are from 10 to 50 minutes. It is highly preferred that an LCD display does NOT show any CO concentration for the first 59 minutes at this concentration to prevent premature WARNING to the end users; hence, reducing false alarm. Therefore, the fact that MICROSIR CO alarm did not display CO concentration for first 4 minutes is actually a good thing. The accuracy of the prototype MICROSIR digital CO alarm was within ±10% in 150-ppm CO after the first 9 minutes.

| | | Prototype MICROSIR |
|---|---|---|
| Elapsed | Reference CO | Digital CO Alarm With |
| Time (Min.) | Conc. (ppm) | S66L Single Sensing Element |
| 0 | 0 | 0 |
| 1 | 120 | 0 |
| 2 | 145 | 0 |
| 3 | 150 | 0 |
| 4 | 150 | 0 |
| 5 | 150 | 101 |
| 6 | 149 | 101 |
| 7 | 150 | 126 |
| 8 | 151 | 163 |
| 9 | 151 | 163 |
| 10 | 151 | 143 |
| 11 | 151 | 145 |
| 12 | 151 | 146 |
| 13 | 150 | 143 |
| 14 | 150 | 137 |
| 15 | 151 | 137 |
| 16 | 150 | 153 |
| 17 | 150 | 153 |

Table 10A & 10B

Electrical Rating or Response Outputs in volt per hour (Table 10A) or in [(Percent Light Obscuration per Hour (% Obs/hr), TABLE 10B] of mini-sized sensing elements type S66 (single sensing element: Models M1-01 & M3-02) and S34 (two sensing elements: Models M1-02 and M3-02) to 0, 15, 70, 150, and 400 ppm CO at Ambient Relative Humidity/Ambient Temperature of 50±20% RH and 23±3° C. These MICROSIR CO Sensor Models were approved by UL on Jan. 17, 2007 as UL Recognized Component: FTAM2 “GAS AND VAPOR DETECTORS AND SENSORS,” File E186246 Vol. 3, Sec. 1. All 4 Models undertook a 1-year -stability study with a constant exposure to 15±3 ppm CO in air at 50±20% RH and 23±3° C. The response output to 70, 150, and 400 ppm CO were measured at 50±20% RH and 23±3° C. before and after the 1-year exposure to 15 ppm CO.

TABLE 10A
MICROSIR Response Output in Voltage Change in Volt per Hour
TABLE 10A. MICROSIR Response Output in Voltage Change in Volt per Hour

| | 0 PPM | 15 PPM | 70 PPM | 150 PPM | 400 |
|---|---|---|---|---|---|
| Model | Volt/hr | volt/hr. | volt/hr. | volt/hr. | volt/hr. |
| M1-01 | (0.004)-0.007 | (0.002)-0.0083 | 0.11-2.54 | 0.70-10.45 | 1.75-29.09 |
| M1-02 | (0.004)-0.007 | (0.002)-0.0083 | 0.01-0.78 | 0.25-8.40 | 0.91-31.48 |
| M3-01 | (0.004)-0.007 | (0.002)-0.0083 | 0.07-3.10 | 0.61-10.43 | 0.99-36.10 |
| M3-02 | (0.004)-0.007 | (0.002)-0.0083 | 0.004-0.99 | 0.44-7.55 | 1.57-28.80 |

TABLE 10B
MICROSIR Response Output in Percent Light Obscuration per Hour (% Obs/hr)

| | 0 PPM | 15 PPM | 70 PPM | 150 PPM | 400 |
|---|---|---|---|---|---|
| Model | % Obs/hr | % Obs/hr | % Obs/hr | % Obs/hr | % Obs/hr |
| M1-01 | (0.18)-0.45 | (0.065)-0.28 | 3.7-84.83 | 23.50-348.39 | 52.37-969.74 |
| M1-02 | (0.14)-0.45 | (0.065)-0.28 | 0.24-26.02 | 8.24-279.85 | 30.46-1049.30 |
| M3-01 | (0.14)-0.45 | (0.65)-0.28 | 2.25-103.17 | 20.28-347.63 | 32.96-1203.30 |
| M3-02 | (0.14)-0.45 | (0.65)-0.28 | 0.13-33.06 | 14.75-251.81 | 52.25-959.94 |

### DETAILED DESCRIPTION OF DRAWINGS

FIG. 1 is an assembly drawing of MICROSIR MOD3-01 (M3-01) system 100, which comprises the reservoir assembly 101, the sensor housing 106 with the housing portions 106a and 106b, a connecting gas opening 126 therebetween, gas diffusion holes 124, one CO sensing disk 105, shock absorber 104, and getter systems 103 the cap 107, the lens 110, light pipe 108 and light pipe holder plate 109. Located in the interior of the sensor housing 106 is one sensing element 105. The gasket 102 connects and seals the reservoir assembly 101 to the sensor housing 106. The locking ears 120 are used to located and hold the reservoir 101 into the sensor housing 106 by means of a locking groove 122. This housing sits atop a surface mounted LED and Photodiode (not shown), which are mounted on a PC board. The sensor housing is located by two pins 114 and two screws located on the plate 111. Two screws are located at two screw holes 112. The clear plate with lens 110 is welded in place and the light pipe 108 is held in place by the cap 107 (which may be welded) and the light pipe is sealed by an O-ring 113. The clear plate 110 may also be welded and mounted right above the surface mount LED (not Shown). The reservoir assembly 101 comprises a containing unit 121 and a membrane (not shown) sealed to a bottom grid (see FIGS. 7 and 8), which has a number of holes and a the top 115 is welded on to the reservoir assembly 101. Then the reservoir is inserted after small holes (not shown). The chemical content of salt solution and dyes are placed inside the reservoir cylinder 101 and the clear polyethylene top 115 is photon welded to the cylinder, the sensor 105 is placed in the interior chamber of the housing portion 106b. The reservoir is held by locking the ears 120 interfacing with the locking grooves 122. The getter system 103 is placed in the gas-path opening 126 before the sensing element 105. The getter system 103 may comprise materials that remove basic gases as well as other gases and vapors such as those of volatile organic compounds (VOCs). In addition, there is a small opening inside the getter that controls gas path (not Shown). The size of the air quality and humidity controlled chamber within the small hole defined by the small hole on one side and the reservoir on the opposite side, this chamber may also be defined by the O-ring 113 on the light pipe and the lens 110 at the bottom.

FIG. 2 is an assembly drawing of MICROSIR MOD3-02 (M3-02) system 200, which comprises the reservoir assembly 201, the sensor housing 206 with housing portions 206a and 206b, a connecting gas opening therebetween, gas diffusion holes 224, the sensors 205, shock absorber 204, and getter systems 203 the cap 207, the lens 210, light pipe 208 and light pipe holder plate 209. Located in the interior of the sensor housing 206 are two sensing elements 205 for detecting wider range of CO concentrations. The gasket 202 connects and seals the reservoir assembly 201 to the sensor housing 206. The locking ears 220 are used to located and hold the reservoir 201 into the sensor housing 206 by means of a locking groove 222. This housing sits atop a surface mounted LED and Photodiode (not shown), which are mounted on a PC board. The sensor housing is located by two pins 214 and two screws located on the plate 211. Two screws to be located at two screw holes 212. The clear plate with lens 210 is welded in place and the light pipe 208 is held in place by the cap 207 (which may be welded) and the light pipe is sealed by an O-ring 213. The clear plate 210 may also be welded and mounted right above the surface mount LED (not Shown). The reservoir assembly 201 comprises a containing unit 221 and a membrane (not shown) sealed to the bottom grid (see FIGS. 7 and 8), which has a number of holes and then a top 215 is welded on to the reservoir assembly 201. Then the reservoir is inserted after small holes (not shown). The chemical content of salt solution and dyes are placed inside the reservoir cylinder 201 and the clear polyethylene top 215 is photon welded to the cylinder, the sensor 205 is placed in the interior chamber of the housing portion 206b. The reservoir is held by locking the ears 220 interfacing with the locking grooves 222. The getter system 203 is placed in the gas-path opening 226 before the sensing element(s) 205. The getter system 203 may comprise materials that remove basic gases as well as other gases and vapors such as those of volatile organic compounds (VOCs). In addition, there is a small opening inside the getter that controls gas path (not Shown). The size of the air quality and humidity controlled chamber within the small hole defined by the small hole on one side and the reservoir on the opposite side, this chamber may also be defined by the O-ring 213 on the light pipe and the lens 210 at the bottom.

FIG. 3 is an assembly drawing of SIR-01 system 300 showing the reservoir 301 containing MnCl₂ chemical content (not shown), the controlled gas diffusion holes 302, acid impregnated getter felt 303 for removing ammonia/amine, sensor holder 307, ONE sensing element 308, getter+shock absorber sub-assembly 306 for additional protection against ammonia/amine and volatile organic compounds (VOCs), and retainer clip 305 for locking the sensor and the sub-assembly in place. The assembled sensor is installed inside a sensor holder 311, containing a photodiode 310 and a light emitting diode 309. Once the assembled sensor is installed, the getter felt 303 is located on top of the retainer 305; the reservoir 301 is snapped onto the sensor holder 311.

FIG. 4 is an assembly drawing of SIR-02 system 400 showing the reservoir 401 containing MnCl₂ chemical content (not shown), the controlled gas diffusion holes 402, an acid impregnated getter felt 403 for removing ammonia/amine, sensor holder 407, TWO sensing elements 408 for detecting wider concentrations of CO, getter+shock absorber sub-assembly 406 for additional protection against ammonia/amine and volatile organic compounds (VOCs), and retainer clip 405 for locking the sensor and the sub-assembly in place. The assembled sensor is installed inside a sensor holder 411, containing a photodiode 410 and a light emitting diode 409. Once the assembled sensor is installed, the getter felt 403 is located on top of the retainer 405; the reservoir 401 is snapped onto the sensor holder 311.

FIG. 5 is graphical representation of the results shown in Table 8. The result was based on a single sensing element type S66L assembled inside a MICROSIR MOD1 (M1) housing assembly as shown in FIG. 7, which is further assembled onto a PCB boards, which is operated according to a set of instructions as programmed in the software. The accuracy of the digital display of the MICROSIR CO sensing system is within ±13% in 70-ppm CO, when compared to the actual CO concentration.

FIG. 6 is graphical representation of the results shown in Table 9. The result was based on a single sensing element type S66L assembled inside a MICROSIR MOD1 (M1) housing assembly as shown in FIG. 7, which is further assembled onto a PCB boards, which is operated according to a set of instructions as programmed in the software. The accuracy of the digital display of the MICROSIR CO sensing system is within ±10% in 150-ppm CO, when compared to the actual CO concentration.

FIG. 7 is an assembly drawing of MICROSIR MOD1-01 (M1-01) system, which comprises the reservoir assembly 701, the gasket 702, the shock absorbers 704, one mini CO sensing element 705, assembled housing 710, a getter systems 715, the cap 720, the diffusion controlled gas-path 730 in the cap 720. The assembled housing 710 includes housing portions 710a, 710b, 710c. Like the microSIR MOD3 (M3) (FIGS. 1&2), the MOD1 (M1) also contained within the assembled housing, the lens (not shown), light pipe (not shown), and light pipe holder plate (not shown). Located in the interior of the assembled housing 710 is one mini sensing element 705. The gasket 702 connects and seals the reservoir assembly 701 to the assembled sensor housing 710. Like the MOD3 (M3), the MOD1 (M1) also has locking ears to locate and hold the reservoir into the sensor housing by means of a locking groove. The assembled housing sits atop a surface mounted LED (not shown) and Photodiode (not shown), which are mounted on a PC board (not shown). The sensor housing is also located by two pins (not shown) and two screws (not shown) located on the plate. The clear plate with lens (not shown) is welded in place and the light pipe (not shown) is held in place by the plate (not shown) and the light pipe is sealed by an o-ring (not shown). The clear plate (not shown) may also be welded and mounted right above the surface mount LED (not Shown). The reservoir assembly 701 comprises a containing unit 721 and a membrane (not shown) sealed to the bottom grid, which has a number of holes and then the top is welded on to the reservoir assembly 701. The chemical content of salt solution and dyes are placed inside the reservoir cylinder 701 and the clear polyethylene top is photon welded to the cylinder, the sensor 705 is placed in the interior chamber of the assembled housing portion 710b. The reservoir is held by locking the ears interfacing with the locking grooves. The getter system 715 is placed in the gas-path opening 730 before the sensing element 705. The getter system 715 may comprise materials that remove basic gases as well as other gases and vapors such as those of volatile organic compounds (VOCs). In addition, there is a small opening inside the getter that controls gas path (not shown). The size of the air quality and humidity controlled chamber within the small hole defined by the small hole on one side and the reservoir on the opposite side, this chamber may also be defined by the O-ring (not shown) on the light pipe and the lens (not shown) at the bottom.

FIG. 8 is an assembly drawing of MICROSIR MOD1-02 (M1-02) system, which comprises the reservoir assembly 801, the gasket 802, the shock absorbers 804, two mini CO sensing elements 805, assembled housing 810, a getter systems 815, the cap 820, the diffusion controlled gas-path 830. The assembled housing 810 includes housing portions 810a, 810b and 810c. Like the micro SIR MOD3 (FIGS. 1&2), the MOD1 also contained within the assembled housing, the lens (not shown), light pipe (not shown), and light pipe holder plate (not shown). Located in the interior of the assembled housing 810 are two mini sensing elements 805. The gasket 802 connects and seals the reservoir assembly 801 to the assembled sensor housing 810. Like the MOD3, the MOD1 also has locking ears to locate and hold the reservoir into the sensor housing by means of a locking groove. The assembled housing sits atop a surface mounted LED (not shown) and Photodiode (not shown), which are mounted on a PC board (not shown). The sensor housing is also located by two pins (not shown) and two screws (not shown) located on the plate. The clear plate with lens (not shown) is welded in place and the light pipe (not shown) is held in place by the plate (not shown) and the light pipe is sealed by an o-ring (not shown). The clear plate (not shown) may also be welded and mounted right above the surface mount LED (not shown). The reservoir 801 comprises a containing unit 821 and a membrane (not shown) sealed to the bottom grid, which has a number of holes and then the top is welded on to the reservoir. The chemical content of salt solution and dyes are placed inside the reservoir cylinder 801 and the clear polyethylene top is photon welded to the cylinder, the sensors 805 are placed in the interior chamber of assembled housing 810b. The reservoir is held by locking the ears interfacing with the locking grooves. The getter system 815 is placed in the gas-path opening 830 before the sensing element 805. The getter system 815 may comprise materials that remove basic gases as well as other gases and vapors such as those of volatile organic compounds (VOCs). In addition, there is a small opening inside the getter that controls gas path (not shown). The size of the air quality and humidity controlled chamber within the small hole defined by the small hole on one side and the reservoir on the opposite side, this chamber may also be defined by the O-ring (not shown) on the light pipe and the lens (not shown) at the bottom.

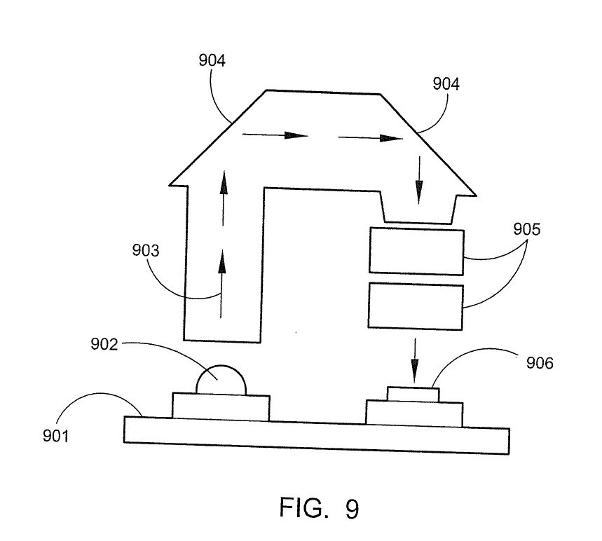

FIG. 9 is a side-view illustration of the theory of operation for the MICROSIR CO sensing system. This illustration explains the “Theory of Operation” of the MICROSIR Sensing System. Shown in the illustration is the PCB board 901, the IRLED 902, the light pipe 903 and its defective turns 904, the sensing elements (ONE or TWO, shown are TWO), and the photo detector 905. The response characteristic (output) of the MICROSIR CO Sensor 905 is the measure of light obscuration 903 through the semi-transparent MICROSIR CO Sensing element(s) 905. Like Quantum's current, large-sized SIR CO sensors, the new MICROSIR CO sensors are also highly selective to CO. When the sensing element(s) 905 encounters CO (not shown), it darkens (not shown). When CO is removed, the sensor returns to its original state (recovery, not shown). The darkening rate of the sensor is proportional to CO gas concentration in the air surrounding the sensor. To monitor the sensing element's rate of darkening (sensor+CO reaction) and/or lightening (recovery), a light source such as an Infrared Light Emitting Diode (IRLED) 902 pulses or emits photons 903 every 30 to 45 seconds. The emitting photons 903 journey are guided by the light pipe and its turns 904 to the sensing element(s) 905. The existing protons are then detected by a photodiode 906. The higher the CO concentration reacting with the sensor, the darker the sensing element(s), the fewer the number of photons (amount of light) detected by the photodiode.

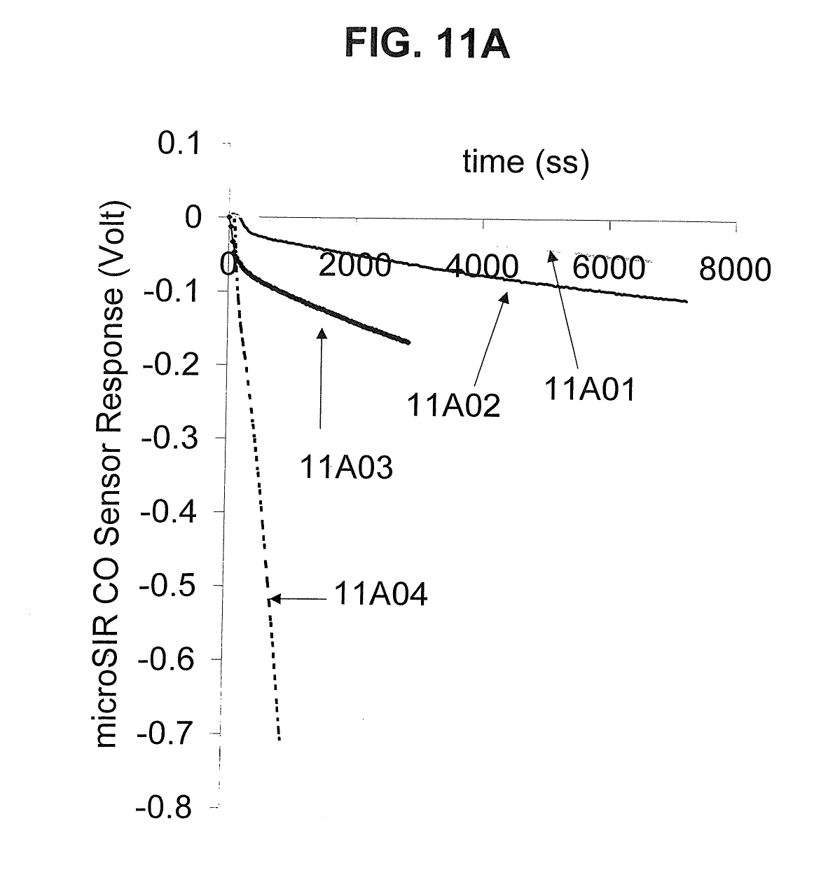

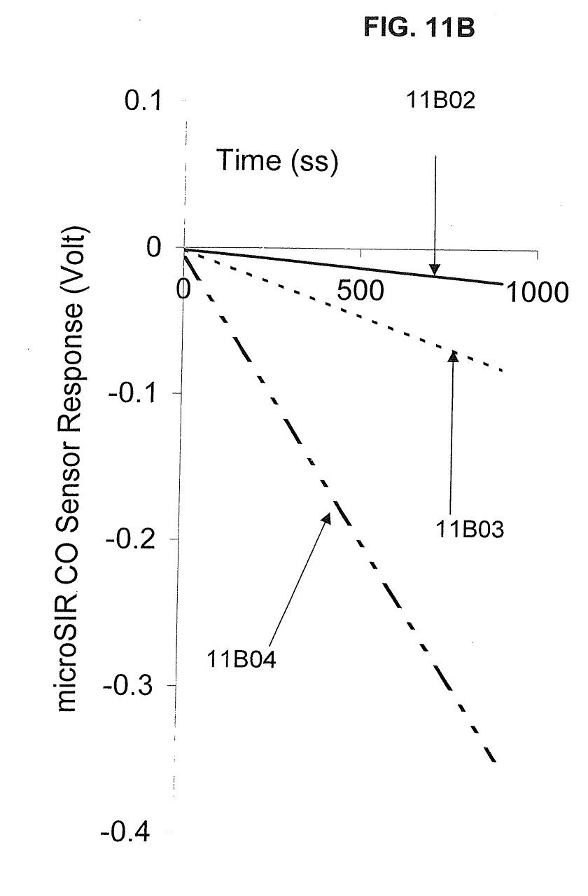

FIG. 10 is a graphical representation showing the response characteristics of ONE mini-sized S66 sensor series, in a MICROSIR MOD1-01 to, 70 ppm 1002, 150 ppm 1003, and 400 ppm CO 1004 at 23±3° C. and 55±5% RH, as specified in criteria 1. Sensing elements were singly installed in the MICROSIR MOD1-01 assembly configuration (FIG. 7) then mounted on the 8UP-MICROSIR-voltage output board, so the sensor output is converted to a voltage level corresponding to the obscuration of light passing through the MICROSIR CO sensing element. The signal conditioning is performed by a test circuit containing an operational amplifier (OpAmp). The amplification circuit is set to attain an initial value of 4 Volts output. As the sensor responds to CO, the voltage output decreases. This voltage-output board is a subject of a co-pending U.S. Patent application No. 60/711,748, filed on Aug. 25, 2005. The complete assembled samples were then stored inside a Thermotron environmental chamber, which maintained at 23° C./55% RH. CO was injected into the chamber to create and maintain 30±3 ppm for 8 hours, 70±3 ppm for 4 hours, 150±5 ppm for 50 minutes, and 400±10 ppm for 15 minutes. At the end of each CO gas test, air injection was necessary to purge out the CO and to regenerate the sensing element for the next CO gas test. The responses are expressed as change in the voltage output (volt) versus time. The responses are as expected. That is, the high the CO concentration the bigger the responses. Following this test, the system is subjected “sequentially” to selected tests at extreme conditions as described in FIGS. 11 through 14 to verity the system performance to the UL 2034 standards for both the RESIDENTIAL and RECREATIONAL VEHICLE requirement.

FIG. 11A is a graphical representation showing the response characteristics of the same MICROSIR CO sensor system from FIGS. 10 to 30 ppm 11A01, 70 ppm 11A02, 150 ppm 11A03, and 400 ppm CO 11A04 at 49° C. and 40% RH, as specified in criteria 6. The system was preconditioned at 49° C./40% RH for 3 hours prior to the CO exposures at the same conditions. There is a clear differentiation among the responses to four different CO concentrations ranging from 30 to 400 ppm. Following the 49° C./40% RH test, the system was subjected to a 66° C./40% RH as described in FIG. 11B.

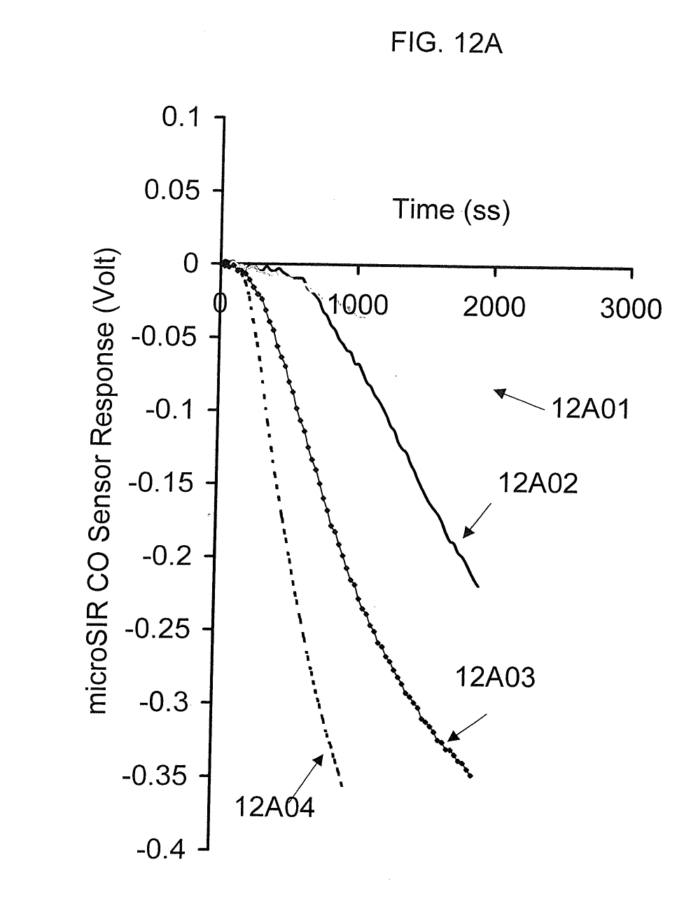

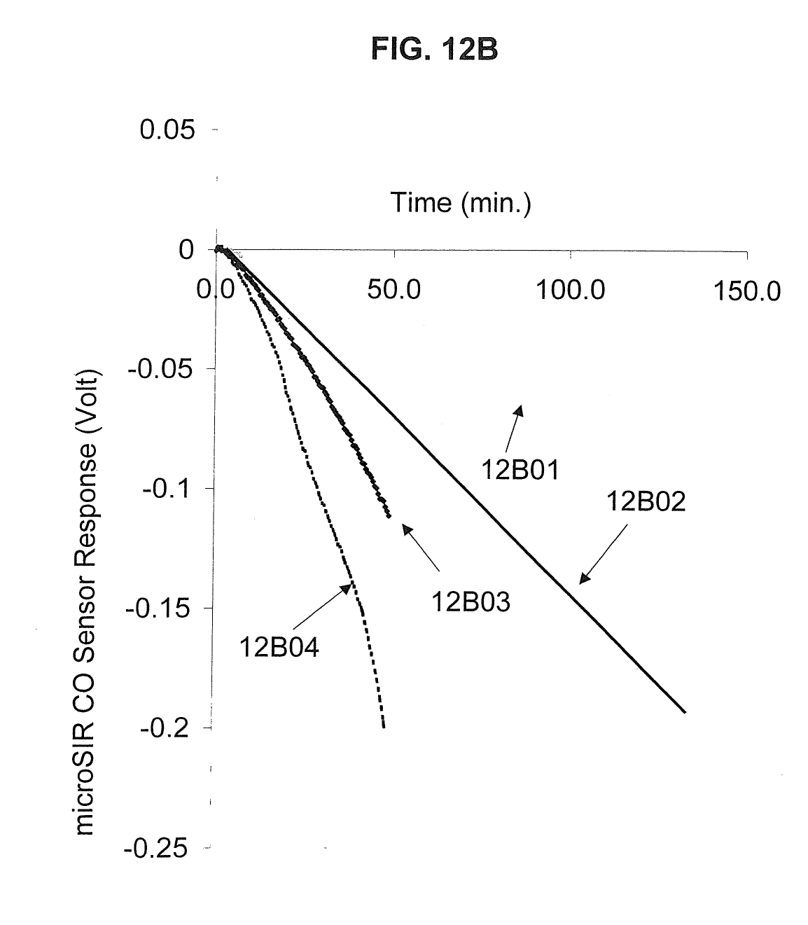

FIG. 11B is a graphical representation showing the response characteristics of the same MICROSIR CO sensor system from FIG. 11A to 70 ppm 11B02, 150 ppm 11B03, and 400 ppm CO 11B04 at 66° C. and 40% RH, as specified in UL 2034 Section 69.1a. The system was preconditioned at 66° C. and 40% relative humidity for 30 days prior to the CO exposures at the same conditions. There is a clear differentiation among the responses to three different CO concentrations ranging from 70 to 400 ppm. The response to 30 ppm (not shown) was not measured but is expected to have the least voltage change. Following the 66° C./40% RH test, the system was subjected to 0° C./15% RH as described in FIG. 12A.

FIG. 12A is a graphical representation showing the response characteristics of the same MICROSIR CO sensor system from FIG. 11B to 30 ppm 12A01, 70 ppm 12A02, 150 ppm 12A03, and 400 ppm CO 12A04 at 0 C.° C. and 15% RH, as specified in Criterion 7 or UL 2034 Section 45. The system was preconditioned or stored at 0° C./15% RH for 3 hours prior to the CO exposures at the same conditions. There is a clear differentiation among the responses to all four different CO concentrations ranging from 30 to 400 ppm. Following the 0° C./15% RH test, the system was subjected to a minus (−) 40C test as described in FIG. 12B.

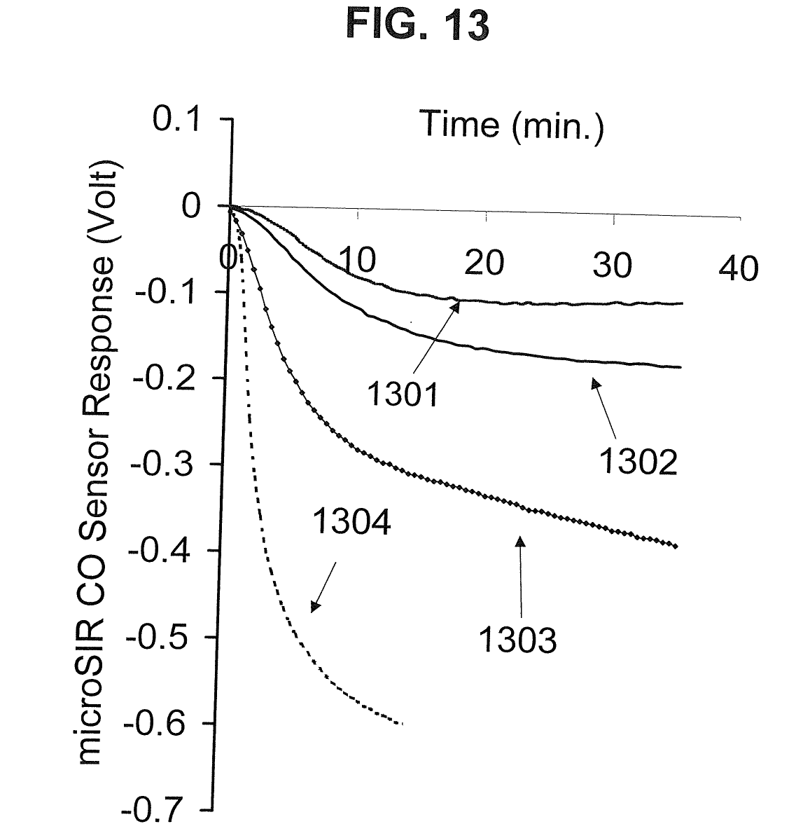

FIG. 12B is a graphical representation showing the response characteristics of the same MICROSIR CO sensor system from FIG. 12A to 30 ppm 12B01, 70 ppm 12B02, 150 ppm 12B03, and 400 ppm CO 12B04 at minus (−) 40 C.°, as specified in UL 2034 Section 69.1b. The system was preconditioned or stored at minus (−) 40° C. for 3 days prior to the CO exposures at the same conditions. There is a clear differentiation among the responses to four different CO concentrations ranging from 30 to 400 ppm. Following the minus (−) 40° C. test, the system was subjected to a minus 61° C./93% RH as described in FIG. 13.

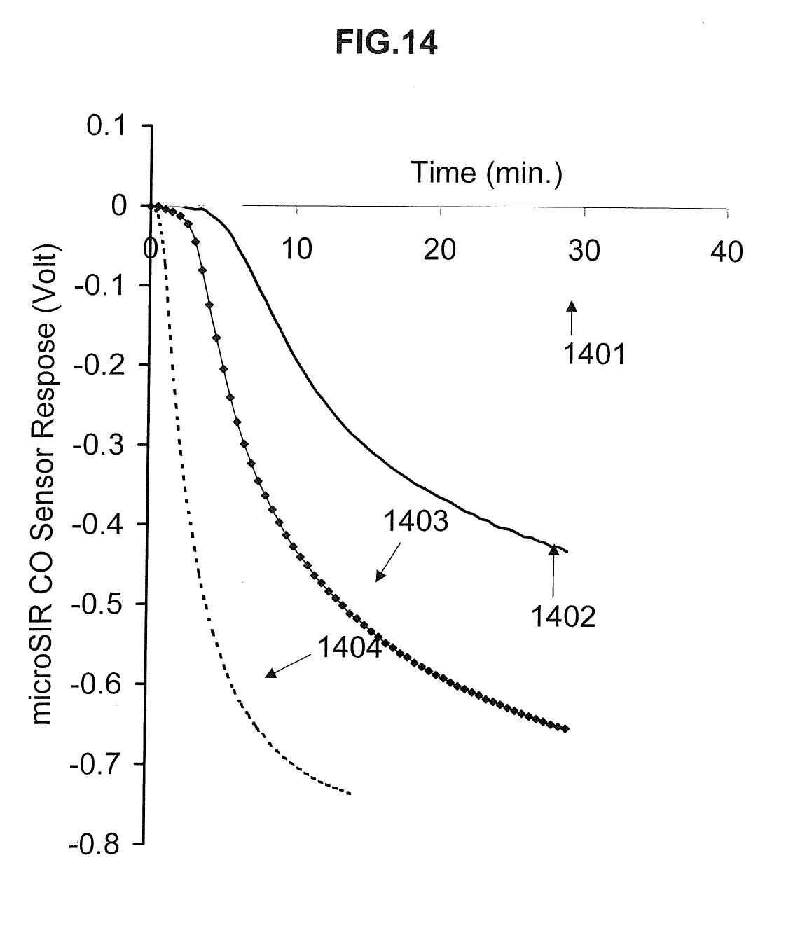

FIG. 13 is a graphical representation showing the response characteristics of the same MICROSIR CO sensor system from FIG. 12B to 30 ppm 1301, 70 ppm, 150 ppm 1303, and 400 ppm CO 1304 at minus 61° C. and 93% RH, as specified in UL 2034 Section 69.1c. The system was preconditioned or stored at 61° C./93% RH for 10 days prior to the CO exposures at the same conditions. There is a clear differentiation among the responses to four different CO concentrations ranging from 30 to 400 ppm. Following the 61° C./93% RH test, the system was subjected to a minus 23° C./10% RH as described in FIG. 14.

FIG. 14 is a graphical representation showing the response characteristics of the same MICROSIR CO sensor system from FIG. 13 to 30 ppm, 70 ppm 1402, 150 ppm 1403, and 400 ppm CO 1404 at 23° C. and 10% RH, as specified in UL 2034 Section 46A.2. The system was preconditioned or stored at 23° C./10% RH for 7 days prior to the CO exposures at the same conditions. Again, there is a clear differentiation among the responses to four different CO concentrations ranging from 30 to 400 ppm. This test concluded the required “Sequential” CO performance required as specified in UL 2034, Section 41.3 for both the “conditioned and unconditioned areas” applications. FIGS. 10 through 14 clearly show that the ONE mini-sized CO sensor in the MICROSIR CO sensing system can meet all performance criteria necessary for obtaining the UL 2034 approval for both the Residential (conditioned) and Recreational Vehicle (unconditioned) approval.

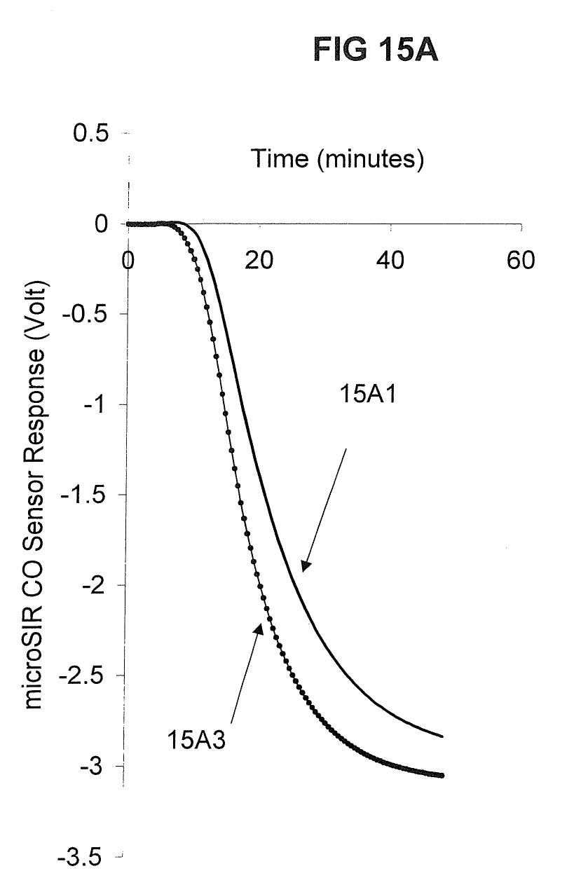

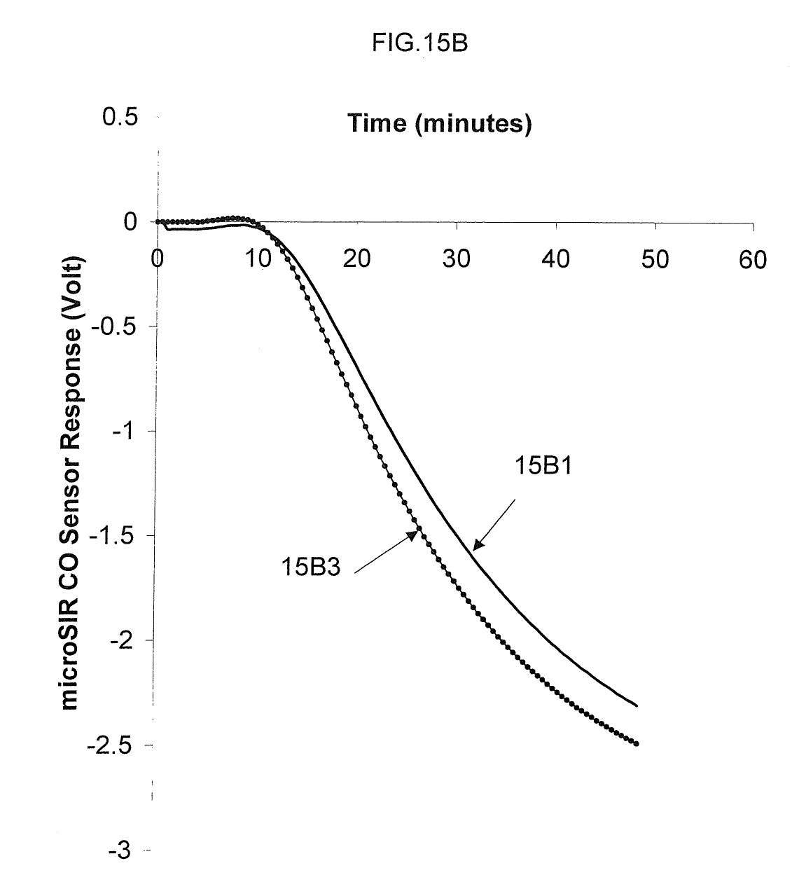

FIG. 15A is a graphical representation showing the comparative response characteristics of ONE mini-sized S66 sensor series to 150 ppm CO in a MICROSIR MOD1-01 15A1 versus in a MICROSIR MOD3-01 15A3 at 23±3° C. and 55±5% RH. The dash 01 following a MOD identifies that there is ONLY ONE sensing element. Like those samples in FIGS. 10 to 14, these assembled samples were also mounted on the same type of 8UP-MICROSIR-voltage output board for this test. CO injection and air purge were done in the same manners as described in FIG. 10. The results were also analyzed and presented in the same manner. FIG. 15A indicates that given the same identical sensor formulation in the same CO test, the magnitude of response is greater when that sensor formulation is installed in the MOD3 15A3 than when it is installed in the MOD1 15A1.

FIG. 15B is a graphical representation showing the comparative response characteristics of TWO mini-sized S34 sensor series to 150 ppm CO in a MICROSIR MOD1-02 15B1 versus in a MICROSIR MOD3-02 15A3 at 23±3° C. and 55±5% RH. The dash 02 following a MOD identifies that there are TWO sensing elements. Like those samples in FIGS. 10 to 14, these assembled samples were also mounted on the same type of 8UP-MICROSIR-voltage output board for this test. CO injection and air purge were done in the same manners as described in FIG. 10. The results were also analyzed and presented in the same manner. FIG. 15B indicates that given the same identical sensor formulation in the same CO test, the magnitude of response is greater when that sensor formulation is installed in the MOD3 15B3 than when it is installed in the MOD1 15B1. FIG. 15B is in agreement with FIG. 15A.

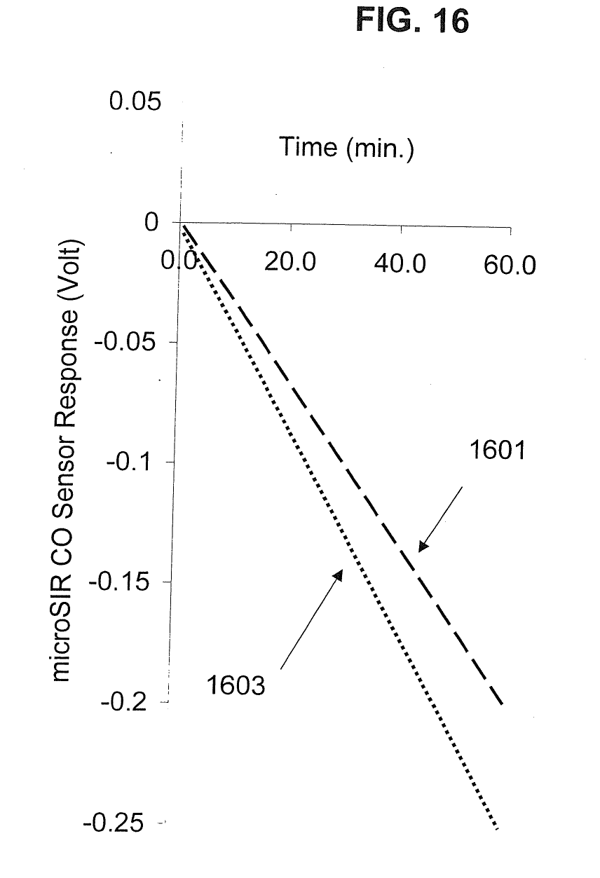

FIG. 16 is a graphical representation showing the comparative response characteristics of ONE mini-sized S66 sensor series to 150 ppm CO in a MICROSIR MOD1-01 1601 versus in a MICROSIR MOD3-01 1603 at 66° C./40% RH following a 30 days of preconditioning at 66° C./40% RH. Like those samples in FIGS. 10 to 14, these assembled samples were also mounted on the same type of 8UP-MICROSIR-voltage output board for this test. CO injection and air purge were done in the same manners as described in FIG. 10. The results were also analyzed and presented in the same manner. FIG. 16 indicates that given the same identical sensor formulation in the same CO test, the magnitude of response is greater when that sensor formulation is installed in the MOD3 1603 than when it is installed in the MOD1 1601. FIG. 16 is in agreement with FIGS. 15A and 15B

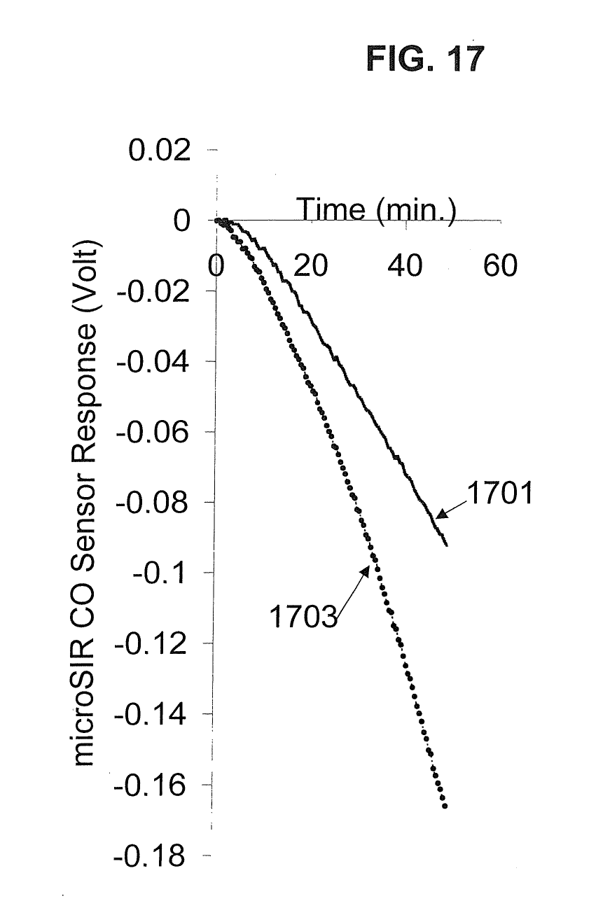

FIG. 17 is a graphical representation showing the comparative response characteristics of ONE mini-sized CO sensor from the S66 sensor series to 150 ppm CO in a MICROSIR MOD1-01 1701 versus in a MICROSIR MOD3-01 1703 at minus (−) 40° C. following a 3 days of preconditioning at (−) 40° C. Like those samples in FIGS. 10 to 14, these assembled samples were also mounted on the same type of 8UP-MICROSIR-voltage output board for this test. CO injection and air purge were done in the same manners as described in FIG. 10. The results were also analyzed and presented in the same manner. FIG. 16 indicates that given the same identical sensor formulation in the same CO test, the magnitude of response is greater when that sensor formulation is installed in the MOD3 1703 than when it is installed in the MOD1 1701. FIG. 17 is in agreement with FIGS. 15A and 15B and 16. That is according to FIGS. 15 through 17, the same identical sensor formulation is always faster in responding to same CO concentration within the same test, from −40° C. to +66° C., when it is installed in a MOD3 than when it is installed in a MOD1. These figures also show that both MOD1 and MOD3 are capable of meeting the UL 2034 requirement for both residential and recreational vehicle approval.

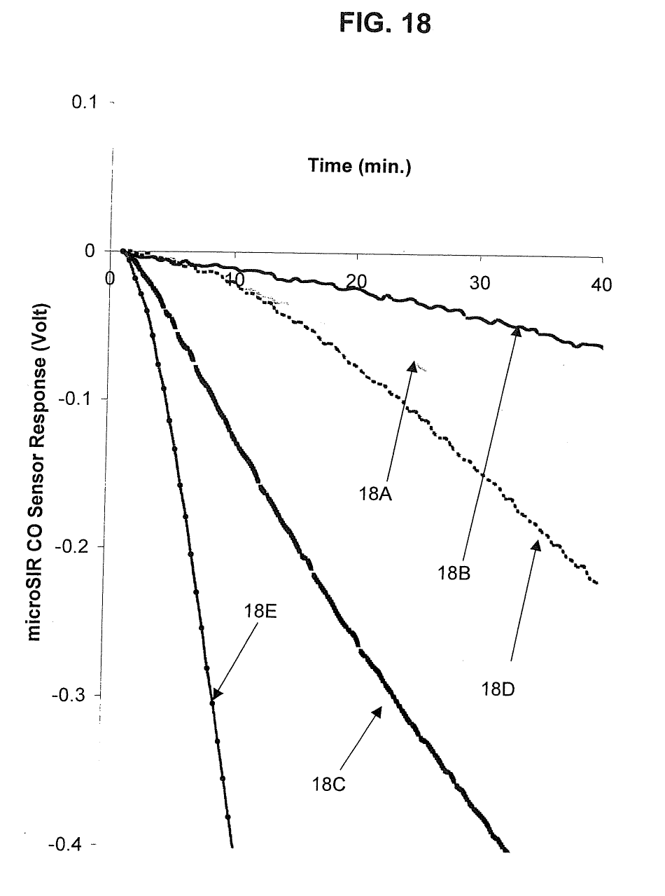

FIG. 18 is a graphical representation showing the IMPROVED response characteristics of the ONE mini-sized S6 sensor formulations with CaCl₂ partially to completely replaced by various proportions of ZnCl₂ and ZnBr₂ as follows: 100% CaCl₂ 18A (control), 100% ZnCl₂ 18B, 50% ZnCl₂+50% ZnBr₂ 18C, 50% CaCl₂+50% ZnCl₂ 18D, and 50% CaCl₂+50% ZnBr₂ 18E. The samples were singly installed in a MICROSIR MOD1-01. Like those samples in FIGS. 10 to 14, these assembled samples were also mounted on the same type of 8UP-MICROSIR-voltage output board for this test. The samples were preconditioned at 66° C./40% RH for 30 days. At the end of the 30^{th} day, CO injection to create and maintain 150±5 ppm for 50 minutes while at the 66° C./40% RH before air was introduced to purge CO out. The results were also analyzed and presented in the same manner as described in FIG. 10. FIG. 18 shows that when CaCl2 is replaced 100% by ZnCl₂ 18B₂, the response is actually lesser than the control with 100% CaCl₂ 18A. The proportion with 50% CaCl₂ and 50% ZnBr₂ 18E is the most sensitive one among all 5. Following this test, the sampled was tested at −40 C (not shown), but due to bad electronic noise, no valid results were obtained. Following the −40 C test, the samples were subjected to a 61° C./93% RH (not shown), where corrosion on some test sites prohibited the measurement of most sensor formulations, except those of the control (100% ZnCl₂) and the 50% ZnCl₂+50% ZnBr₂ formulation. That result showed that the 50% ZnCl₂+50% ZnBr₂ formulation is six times more sensitive than the control (not shown).

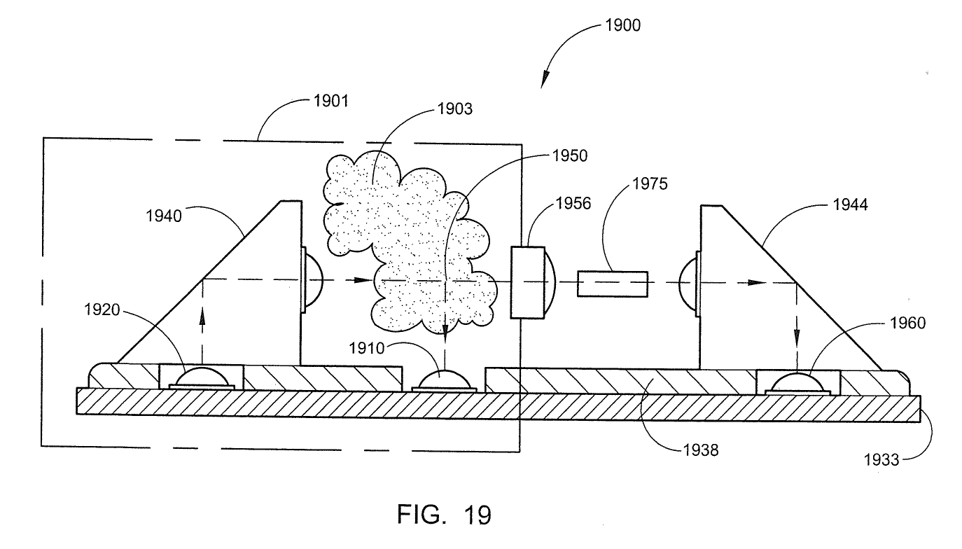

FIG. 19 is an illustration showing ONE MICROSIR CO sensing element (1975) positioned in edge-view orientation for increase sensitivity to low CO concentration for aiding in early fire and/or smoke (1903) detection application. The smoke 1903 enters the chamber and some of the particles scatter photons from the LED 1920, which passes through the prism 1940 before hit the smoke particles. Some of the photons 1950 are scattered at 90 degrees and hit the photodiode 1910, which can be used to trigger an alarm if the threshold of smoke is reached such as 5% smoke. Other photons 1950 continue straight though a lens or window 1956 and then pass through the sensor 1975. Some of the photons 1950 are absorbed proportional to the CO hazard and these remaining photon 1950 pass through a second prism 1944 and are monitored by a second photodiode 1960. The signals of CO and smoke may be combined such that the CO sensing of 20 ppm can make the smoke sensor threshold change to a more sensitive reading such as 4%. In addition, if the CO rises rapidly to some level for example 40 PPM then the smoke may be made even more sensitive to some lower levels such as 2% smoke obscuration. The smoke chamber is open to smoke but not light. Vents (no shown) are used to block the light from entering and let the smoke go in to the smoke chamber 1901. The CO chamber will be sealed from the air and smoke entry using a diffusion type getter (not shown). The getter system is the subject of other patents such as U.S. Pat. No. 6,251,344 B1.

The LED 1920 and the photodiodes 1910 and 1960 are surface mount type and are fixed to the printed circuit board 1933.

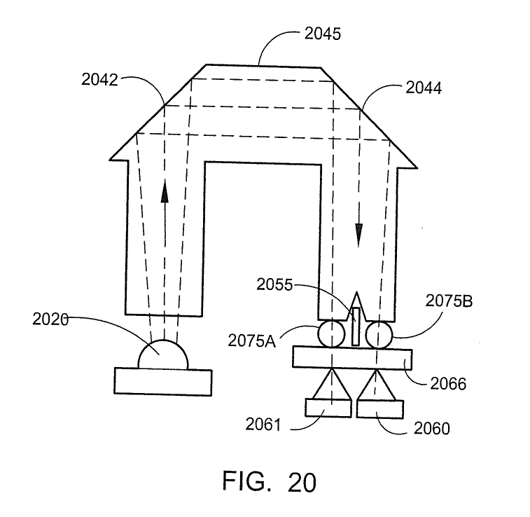

FIG. 20 is an illustration for explaining the “Theory of Operation of MICROSIR involving TWO sensing elements positioned in edge-view orientation” for increase sensitivity within a wider range of humidity and temperature. The LED 2020 is surface mount type and fixed to the PC board not shown. The photons 2030 are emitted from the LED 2020 and travel through the lightpipe 2045 as shown reflecting off of surface 2042 and 2044. The photons 2030 travel either side of the window 2055 and pass through sensing element 2075A and 2075B. Some of the photons are absorbed and other photons continue through the window 2066 and strike either photodiode 2061 or 2060. The photodiode measure the CO hazard and the signal is given to a microprocessor not shown. The circuit and the micro provide an alarm not shown.

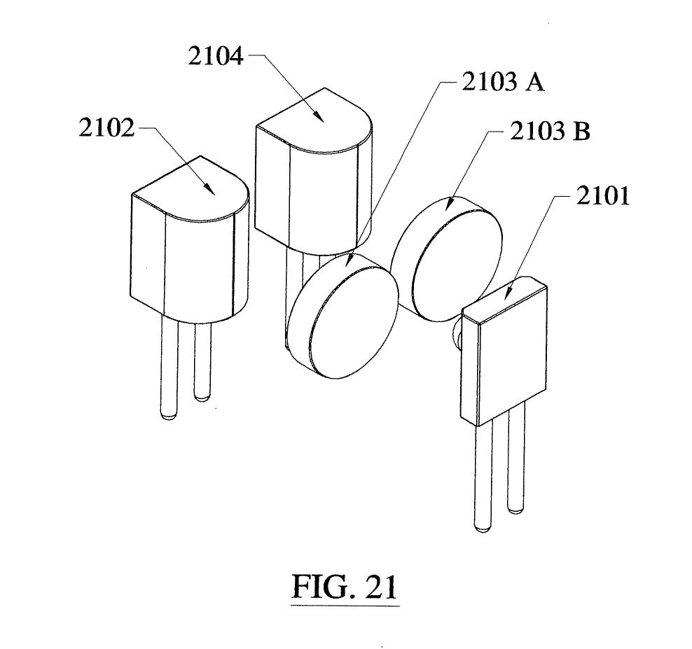

FIG. 21 is an illustration showing two CO sensing elements (2103 A and B) in center-view orientation between one LED 2101 and two photodiodes 2104 and 2102. One advantage of this system as shown in FIG. 21 is that one sensor may have a high threshold and one a lower level response to provide both fast response and fast regeneration not shown. The sensors 2103 A and 2103 B will regenerate at different speeds. The LED 2101 emits photons (not shown) that pass through both sensing elements 2103A and B and Strike the photodiode 2102 or 2104 where sensor 2103 A is more sensitive to CO it will respond first. As some of the photons are absorbed by 2103A the photodiode measure the CO hazard with the aid of the circuit and microprocessor not shown. The alarm can be sounded by reaction from one or both sensors 2103 A and B. When it is cold the sensor 2103 A regenerates slowly; however, 2103 B regenerates much faster. Therefore the logic circuit uses the fats regenerating sensor not shown. In this way the sensor arrangement can pass the new European standard.

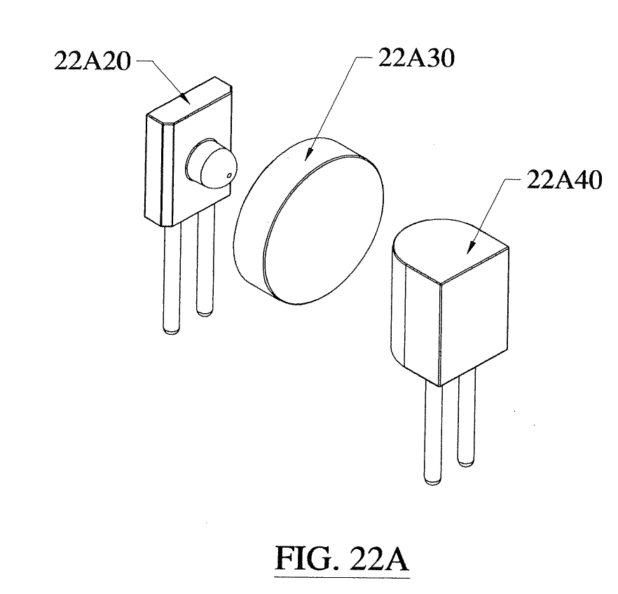

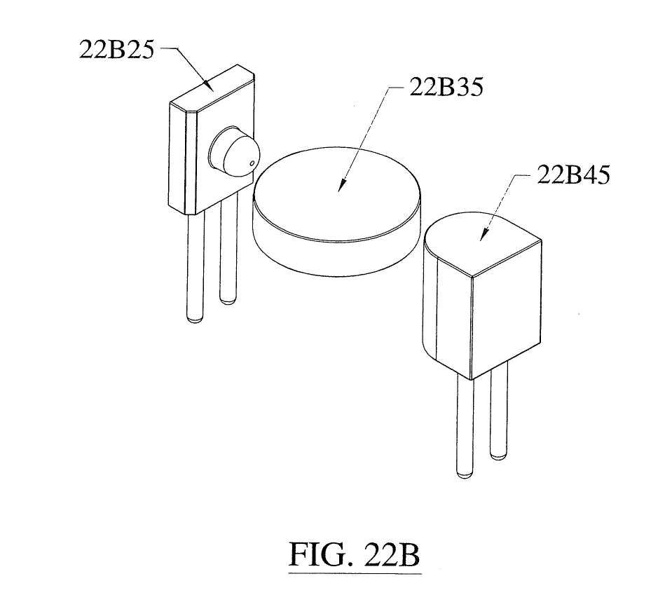

FIG. 22A is an illustration for explaining the “Theory of Operation of SIR-01,” one sensing element 22A30 positioned in center-view orientation” between an LED 22A20 and a Photodiode 22A40. The LED 22A20 emits photons not shown. The photons pass through the center of the sensing elements 22A30 where if CO is present (not Shown) causes the photon to be absorbed. Some photons continue to the photodiode 22A40. The circuit not shown then measure the amount of infrared photons and with the help of the software in the microprocessor (not shown) calculates if there is a need fore alarm and then if so actuate the alarm beeper not shown.

FIG. 22B is an illustration for explaining the “Theory of Operation of SIR-01,” one sensing element 22B35 is positioned in edge-view orientation” between the LED 22B25 and the Photodiode 22B45. This arrange is very useful for passing the Japanese standard and for sensing fires in combination with smoke to produce a enhanced fire detection device or alarm. The sensor changes more rapidly such that a sensor can respond to 550 PPM in 30 seconds. In addition test were conduct in various fires and it was found that each fire test being a standard European fire test produce CO such that the sensor could detect it.

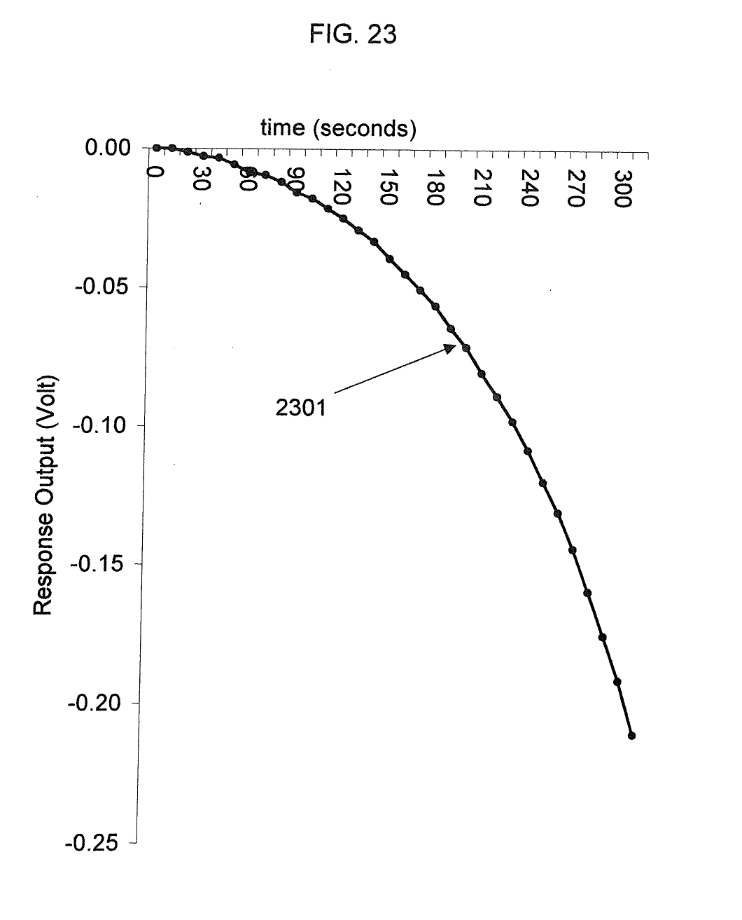

FIG. 23 is a graphical representation showing a response characteristic of ONE mini-sized CO sensor from the S50 sensor series (2301) to a rise in CO ramping of 5-ppm every 30 seconds from 0 to 40 ppm CO ppm. The mini-sized sensing element was prepared according to example 11 (preferred embodiment 10) and was positioned in an edge-view orientation similar to that, which is depicted in FIG. 22B (22B35), or exactly as depicted in FIG. 1 but with the sensing element 105 (FIG. 1) rotated 90°. This assembly construction is to referred as M1-01e (e=edge-view orientation) at 50±20% RH and 23±3° C. Like those samples in FIGS. 10 to 14, the assembled samples were also mounted on the same type of 8UP-MICROSIR-voltage output board for this test. CO was injected at a rate of 5 ppm per 30 seconds to 40 ppm. Clearly, the S50 sensor M1-01e Model can detect CO rising at rate of 5 ppm per every 30 seconds. This is good for early fire detection and elimination of false alarm. The elimination of false alarm comes about by using the input from both CO and particulate and even optionally heat. The percent obscuration of say 6% is detected as a fire with a CO ramp to 15 to 20 PPM in 2 minutes. If the CO is 30 PPM then one can go off earlier by make the logic point of obscuration 5%. In addition if CO is rising rapidly to 40 PPM then the software logic allow alarm at 4% obscuration and so on.

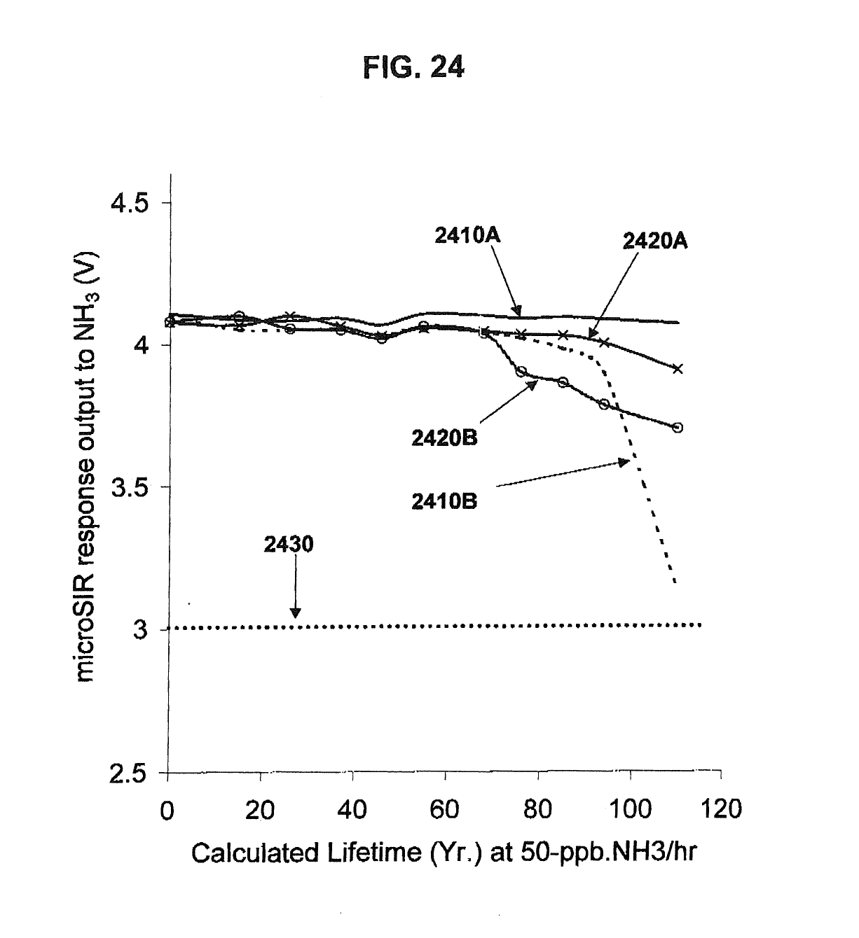

FIG. 24 is graphical representation showing IMPROVED resistance to ammonia damage in M1-01 and M3-01 MICROSIR systems with respect to varying amount of acid-coated activated charcoal sensor used. Ammonia is a known killer of both MICROSIR and SIR CO sensors. Therefore, both the SIR and MICROSIR systems are equipped with getter systems to remove ammonia and/or amine from the incoming air sample (730 of FIG. 7) before it reaches the sensor. For M1-01 MICROSIR system, this improved getter system is 715 of FIG. 7, which is placed in the gas-path opening before the sensing element 705. The getter system 715 may comprise materials that remove basic gases such a ammonia/amine as well as other gases and vapors such as those of volatile organic compounds (VOCs). In the case of M3-01 MICROSIR system, the getter system is 103 of FIG. 1. Like those samples reported in FIGS. 10 to 14, the assembled samples were also mounted on the same type of 8UP-MICROSIR-voltage output board for this test. However, NH3 gas exposed to the samples instead of CO. Since ammonia is the sensor killer, the reverse response is desirable. That is, better getter systems are ones that lead to longer time for sensor-ammonia response output to reach a predetermined sensor end-of-life 2430 of FIG. 24. Give the same type and amount of ammonia getter material, M1-01 MICROSIR systems (2410A, 2410B) are better than M3-01 (2420A, 2410B) systems. 2410A and 2420A contain the same amount and type of getter material. Likewise, 2410B and 2420B also the same amount and same type of getter material. 2410A and 2420A (0.08 g each) contained almost twice the mount that of 2410B and 2420B (0.15 g each). The getter better used was 10% porous activated charcoal beads (0.65-0.85 mm diameters, coated with 10-13% H₃PO₄ by weight) However, it appears that the design of the M1 housing utilizes the getter material more efficiently than does that of M3. In the SIR-1 (not shown) and SIR-02 (not shown) systems, 0.08 g acid-coated have been shown to last ˜60 to 80 years at same 50-ppb.NH₃.Hr⁻¹ ammonia background.

Many other modifications and variations will be apparent to those skilled in the art, and it is therefore, to be understood that within the scope of the appended claims the invention may be practiced otherwise than as specifically described. Some of the current competitive products on the market, which are battery-operated use electrochemical cells for sensors. They are expensive, require frequent calibration because of tendency to drift, respond to interference gases causing many false alarms and have a short stable life. Some models using PEM membranes do not operate below zero and other EC cells use sulfuric acid, which cause corrosive gases to be emitted in hot conditions. Metal Oxide Semiconductor sensors are another competitive technology used in CO alarm on the market today, the MOS sensor take very large amounts of power and therefore cannot be operated practical for most portable applications or for systems. Therefore, there is a need for a low cost, reliable, accurate, easy to use very low powered unit to detect CO level, as well as rate of change of the CO to low level for fire detection, to meet CO standards of various countries such UL 2034 and UL 2075 in the USA and CAS 6.19-01 in Canada. The low cost MICROSIR can meet these standards at cost that are very competitive with MOS and EC sensor technology and perform better, more reliably and with much few false alarms.

What is claimed is:

1. A device for sensing carbon monoxide:
a housing having a first housing portion, a second housing portion and a connecting gas opening therebetween, the first housing portion having at least one gas diffusion hole that allows gas outside the housing to diffuse into the housing;
a surface mounted photon source disposed within the second housing portion;
a surface mounted photon detector disposed within the second housing portion;
at least one sensor element disposed within the second housing portion, the sensor element having an optical property responsive to CO;
a reservoir assembly adapted to control humidity with in the housing, the reservoir assembly attached to the first housing portion and comprising a containing unit with a grid having holes; and
a getter disposed in the connecting gas opening between the reservoir and the sensor element, the getter adapted to remove contaminates from the gas;
wherein the sensor element comprises a substrate with pores having a pore size in the range of about 10 nm to 50 nm, and a coating comprising a supermolecular complex chemical compound comprising molybdenum, palladium, copper, at least one alkaline or transition metal ion, at least one cyclodextrin or cyclodextrin derivative, and at least one acid.

2. A device of claim 1, wherein the sensor element has an expected operational life of about five to fifteen years.

3. A device of claim 1, wherein the photon source emits visible light photons.

4. A device of claim 1, wherein the photon source emits IR photons.

5. A device of claim 1, further comprising means for detecting a change in the optical property of the sensor element.

6. A device of claim 1, wherein the substrate is optically transparent.

7. A device of claim 1, wherein the substrate is about 0.100 inch by 0.025 inch by 0.050 inch.

8. A device of claim 1, wherein the at least one sensor element is a single sensor element.

9. A device of claim 1, wherein the housing is configured such that the gas passes through the at least one gas diffusion hole and then through the getter.

10. A device of claim 1, wherein the housing is configured such that the gas passes the getter before the sensor element.

11. A device for sensing carbon monoxide:
a housing having a first housing portion, a second portion housing portion and a connecting gas opening between the first and second housing portions, the first housing portion having at least one gas diffusion hole that allows gas outside the housing to diffuse into the housing;
a surface mounted photon source disposed within the second housing portion;
a surface mounted photon detector disposed within the second housing portion;
at least one sensor element disposed within the second housing portion, the sensor element having an optical property responsive to CO;
a reservoir assembly adapted to control humidity with in the housing, the reservoir assembly attached to the first housing portion and comprising a containing unit with a grid having holes; and
a getter disposed in the connecting gas opening, the getter adapted to remove contaminates from the gas before the gas reaches the sensor;
wherein the sensor element includes a substrate and a coating, the substrate comprising porous silica, and the coating comprising at least one material from each of the following groups:
Group 1: Palladium salts selected from the group consisting of palladium sulfite, palladium pyrosulfite, palladium chloride, palladium bromide, palladium iodide, palladium perchlorate, CaPdCl4, CaPdBr4, Na2PdCl4, Na2PdBr4, K2PdCl4, K2PdBr4, Na2PdBr4, CaPdClxBry, K2PdBrxCly, Na2PdBrxCly (where x can be 1 to 3 if y is 4 or visa versa), and mixtures of any portion or all of the above;
Group 2: Molybdenum acid or salts selected from the group consisting of silicomolybdic acid, phosphomolybdic acids, and their soluble salts mixed with acid heteropolymolybdates and mixtures of any portion or all of the above;
Group 3: Soluble salts of copper halides, nitrates, and mixtures thereof, copper organometallic compounds that regenerate the palladium such as copper tetrafluoroacetic acid, copper trifluoroacetylacetonate, and other similar copper compounds, and mixtures of any portion or all of the above;
Group 4: Supramolecular complexing molecules selected from the cyclodextrin family including beta, and gamma as well as their soluble derivatives such as hydroxymethyl, hydroxyethyl, and hydroxypropyl beta cyclodextrin and their derivative, and mixtures of any portion or all of the above;
Group 5: Soluble salts of alkaline and alkali halides, and certain transitional metal halides such as manganese, cadmium, cobalt, chromium, nickel, zinc, and other soluble halide such as aluminum; and any mixture thereof;
Group 6: Organic solvent and/or co-solvent and trifluorinated organic anion selected from the group of trichloroacetic acid, or a mixture of trichloroacetic acid with copper trifluoroacetylacetonate; and any mixture thereof;
Group 7: Soluble inorganic acids such as hydrochloric acid, sulfuric acid, sulfurous acid, or a mixture thereof; and
Group 8: Strong oxidizer such as nitric acid and peroxide, or a mixture thereof.

12. A device of claim 11, wherein the coating comprises Groups 1 through 8 materials in the following mole ratio ranges:
Group 1:Group 3=10.19:1 to 16.98:1
Group 2: Group 3=3.04:1 to 5.07:1
Group 4:Group 3=1.04:1 to 1.74:1
Group 5:Group 3=34.11:1 to 56.84:1
Group 6:Group 3=1.07:1 to 1.79:1
Group 7:Group 3=0.004:1 to 0.04:1
Group 8:Group 3=0.04:1 to 0.08:1,
wherein the device senses a CO concentration of about 30 ppm to 550 ppm.

13. A device of claim 11, wherein the coating comprises Groups 1 through 8 materials in the following mole ratio ranges:
Group 2:Group 1=0.20:1 to 0.33:1
Group 3:Group 1=0.10:1 to 4.73:1
Group 4:Group 1=0.05:1 to 0.08:1
Group 5:Group 1=1.75:1 to 2.92:1
Group 6:Group 1=0.00:1 to 0.00:1
Group 7:Group 1=0.62:1 to 1.03:1
Group 8:Group 1=0.70:1 to 1.16:1,
wherein the device sense a CO concentration of about 550 ppm to 10,000 ppm.

14. A device of claim 11, further comprising means for detecting a change in the optical property of the sensor element.

15. A device for sensing carbon monoxide:
a housing having connected first, second and third housing portions, the second housing portion being between the first and third housing portions, third housing portion having at least one gas diffusion hole that allows gas outside the housing to diffuse into the housing;
a surface mounted photon source and a surface mounted photon detector disposed within the second housing portion;
at least one sensor element disposed within the second housing portion, the sensor element having an optical property responsive to CO;
a reservoir assembly adapted to control humidity within the housing, the reservoir assembly attached to the first housing portion; and
a getter disposed in the third housing portion between the at least one gas diffusion hole and the sensor element, the getter adapted to remove contaminates from the gas;
wherein the sensor element comprises a substrate with pores having a pore size in the range of about 10 nm to 50 nm, and a coating comprising a supermolecular complex chemical compound comprising molybdenum, palladium, copper, at least one alkaline or transition metal ion, at least one cyclodextrin or cyclodextrin derivative, and at least one acid.

16. A device of claim 1, wherein the containing unit of the reservoir assembly is cylindrical.

17. A device of claim 1, wherein in the getter is cylindrical.

18. A device of claim 1, wherein the connecting gas opening is cylindrical.

19. A device of claim 1, wherein the first housing is cylindrical.

20. A device of claim 1, further comprising a light pipe in the second housing portion.
